(19) United States
(12) Patent Application Publication
(10) Pub. No.: US 2025/0051286 A1
(43) Pub. Date: Feb. 13, 2025

(54) SMALL MOLECULE INHIBITORS OF PR3 AND HNE AND USES THEREOF

(71) Applicant: ORIXA THERAPEUTICS LLC, Franklin, TN (US)

(72) Inventors: Adam Seth HAEBERLE, Franklin, TN (US); Matteo PANZA, Franklin, TN (US); James Bernard KRAMER, Franklin, TN (US); Zahra ASSAR-NOSSONI, Franklin, TN (US); Matt SIMON, Franklin, TN (US)

(73) Assignee: ORIXA THERAPEUTICS LLC, Franklin, TN (US)

(21) Appl. No.: 18/721,175

(22) PCT Filed: Dec. 16, 2022
(86) PCT No.: PCT/US2022/081874

§ 371 Date: Jun. 17, 2024

Related U.S. Application Data
(60) Provisional application No. 63/290,406, filed on Dec. 16, 2021.

(51) Int. Cl. C07D 233/38; A61K 31/4025; A61K 31/4174; A61K 31/4178; A61K 31/427; A61K 31/433; A61K 31/4439; A61K 31/506; A61K 31/53; A61K 45/06; A61P 1/04; A61P 37/06; A61P 43/00; C07D 207/50; C07D 401/06; C07D 401/12; C07D 401/14; C07D 403/04; C07D 403/12; C07D 403/14; C07D 417/12

(52) U.S. Cl. CPC C07D 233/38; A61K 31/4025; A61K 31/4174; A61K 31/4178; A61K 31/427; A61K 31/433; A61K 31/4439; A61K 31/506; A61K 31/53; A61K 45/06; A61P 1/04; A61P 37/06; A61P 43/00; C07D 207/50; C07D 401/06; C07D 401/12; C07D 401/14; C07D 403/04; C07D 403/12; C07D 403/14; C07D 417/12

(57) ABSTRACT

Disclosed herein are novel small molecules that can be used therapeutically to treat ailments and diseases associated with an over-active or undesired immune response. Specific embodiments include inhibitors of Proteinase 3 (PR3) and Human Neutrophil Elastase (HNE) and methods for their synthesis and use. The agents can be used to treat alpha-1 antitrypsin deficiency and other ailments related to neutrophil activity.

### RELATED APPLICATIONS

This application claims priority to U.S. provisional patent application No. 63/290,406 filed Dec. 16, 2021, the contents of which are incorporated herein by reference.

### FIELD OF THE INVENTION

The invention relates to small molecule therapeutics, and more specifically, it relates to methods for the synthesis and use of small molecule inhibitors of Proteinase 3 (PR3) and Human Neutrophil Elastase (HNE).

### BACKGROUND

Neutrophils are the most abundant leukocytes in plasma. They are the first cells recruited to injury sites in response to pathogen invasion, and they act in the first line of innate immune defense. Proteases are important effectors of neutrophils. They not only contribute directly to microbicidal activity but also function in the proteolytic processing of chemokines, cytokines and receptors.

Polymorphonuclear neutrophils contain at least four serine endopeptidases, namely neutrophil elastase (NE), proteinase 3 (PR3), cathepsin G (CatG), and NSP4, which contribute to the regulation of infection and inflammatory processes. In physiological conditions, endogenous inhibitors including α2-macroglobulin (α2-M), serpins [α1-proteinase inhibitor (α1-PI)], monocyte neutrophil elastase inhibitor (MNEI), α1-antichymotrypsin, and locally produced chelonianins (elafin, SLPI) control excessive proteolytic activity of neutrophilic serine proteinases.

Many therapeutics target inflammation (TNFa, IL23, IL31), B-cells (Rituximab), T-cells (Abatacept) and even eosinophils (Nucala) and yet the neutrophil is the most common white blood cell (WBC), accounting for 40-60% of all white blood cells. The neutrophil pathogen-killing mechanisms are termed neutrophil extracellular traps (NETs)/NETosis. Neutrophils and NETs are responsible for damage and inflammation in autoimmune indications and play an essential role in several autoimmune and autoinflammatory diseases including ANCA-Associated Vasculitis, ulcerative colitis (UC), systemic lupus, erythematosus, rheumatoid arthritis, gout, and inflammatory bowel disease (IBD).

NETs are associated with lung diseases such as cystic fibrosis, chronic obstructive pulmonary disease (COPD), asthma and transfusion-related acute lung injury. NETs are also associated with cardiovascular diseases including early endothelial dysfunction, atherosclerosis, atherothrombosis and venous thrombosis.

The modulatory effects of NETs/NETosis are exemplified by the caspase-independent activation of IL-1β and IL-18 by Neutrophil Elastase (NE), PR3 and CG (see, e.g., Robertson, Young et al. 2006; Guma, Ronacher et al. 2009 and Joosten, Netea et al. 2009) or the conversion of anti-inflammatory progranulin to pro-inflammatory granulin by NE and PRE (see, e.g., Kenssenbrock, 2008). Furthermore, NE has been shown to couple neutrophil-mediated inflammation with the coagulation pathway by cleaving tissue factor pathway inhibitor on Neutrophil Extracellular Traps (NETs). NETs are released by stimulated neutrophils in a specific form of cell death called NETosis. NETosis is hypothesized to represent a new mechanism of innate immunity mediated by neutrophils in response to pathogen invasion (see, e.g., Brinkmann, Reichard et al. 2004 and Remijsen, Kuijpers et al. 2011).

Granulomatosis with polyangiitis (GPA) is a rare autoimmune disorder where the body produces anti-neutrophil cytoplasmic antibodies (ANCA) to the proteinase 3 receptor (PR3) that lead to specific autoimmune vasculitis. There is a readily available and specific PR3-ANCA test for diagnosis. There are an estimated 10,000 to 17,000 GPA patients in the US and double that number in the EU. The mortality rate associated with GPA is as high as 80% in untreated patients, but use of intensive immunosuppressive regimens (e.g., cyclophosphamide, rituximab, methotrexate, azathioprine, long term glucocorticoids) has decreased mortality rates to nearly 10%. However, hospitalization is still common for patients.

The acronym ‘ELK’ is used to describe the clinical involvement of the ear, nose and throat (ENT); lungs; and kidneys. Raynaud's phenomenon is common and sometimes severe enough to cause tissue death of the tips of the fingers and toes. Mortality is still high in GPA. Early deaths are due to active disease (ESRD) with later deaths showing increased risk of death from infection and cardiovascular disease. The disorder can occur at any age, but most often affects individuals of Northern European descent between 40 and 60 years of age. Additional disease states include any indication where PR3 ANCA or NETosis is part of disease pathology.

In Ulcerative Colitis, approximately ⅓ of UC patients are PR3-ANCA positive. PR3-ANCA Positive Ulcerative Colitis represents and distinct and disease set. The neutrophil and NETs play a direct role in inflammation and gut damage in UC. All current therapeutics focus on inhibiting aberrant gut inflammation and immune response. PR3-ANCA positive UC patients are often non-responsive to anti-TNFa therapy and have the highest disease severity. A subset of at least 30% of UC patients are positive for PR3 ANCA. PR3-ANCA positive results display a positive correlation with disease severity. Serum PR3-ANCA is also a predictor of primary nonresponse to glucocorticoids and anti-TNF-α agents in UC. Neutrophil accumulation in the stool of patients correlates with active disease and neutrophil infiltration is associated with severity of disease.

Neutrophil Extracellular Traps (NETs) induce intestinal damage and thrombotic tendency in UC. NET formation is amplified when neutrophils are incubated with ANCA-IgG isolated from active PR3-ANCA-positive IBD patients. ANCA in UC activate neutrophils through Fc receptor stimulation when binding granule PR3 in the same manner as with AAV. In a mouse model of UC (dextran sulfate sodium (DSS)-induced), elafin was successful in reducing inflammation in a dose-dependent manner (elafin inhibits HNE, PR3 and pancreatic elastase). SLPI was also successful, but elafin was statistically better (SLPI inhibits HNE but not PR3).

In Neutrophil-Driven Inflammatory Non-CF Bronchiectasis and Neutrophil-Driven Inflammatory Cystic Fibrosis, neutrophil serine protease knockdown of 50% has shown statistical significance in prevention and delay of exacerbation of the inflammatory conditions. PR3 and HNE are present in the sputum of CF patients and this level has been shown to correlate with inflammation and exacerbation. Treatment with A1PI in CF patients in an exacerbated state of inflammation has been shown to decrease IL6, IL1β and NE activity.

Primary Sclerosing Cholangitis (PSC) is an idiopathic, heterogeneous, cholestatic liver disease that is characterized by persistent, progressive, biliary inflammation and fibrosis. There is no effective medical therapy for this condition. In the United States, approximately 29,000 patients have this disease. Of those, approximately 30% are PR3-ANCA positive.

In the treatment of Alpha-1 Antitrypsin Deficiency, both HNE and PR3 play a role. A small molecule that is capable of inhibiting both neutrophil serine proteases may be beneficial and potentially disease modifying. While HNE is the traditional target of AATD, PR3 has been shown to play a much greater role than initially thought in A1PI deficient COPD and A1PI augmentation leads to a major reduction in the PR3 footprint.

Therefore, an unmet need exists for disease-modifying inflammation reduction via small molecule neutrophil serine protease inhibitors. Disclosed herein are definitions, methods, systems, and compositions for applying the novel facets of the instant invention, allowing one of ordinary skill in the art to lawfully reproduce the invention given the guidance of this disclosure.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

An unwanted immune response can occur in autoimmune disease, transplant rejection and allergy. Embodiments include pharmaceutical agents (e.g., neutrophil serine proteases) and methods of suppressing an undesired immune response.

Embodiments include a compound, or a pharmaceutically acceptable salt thereof, wherein the compound is selected from DCT-5A, DCT-5B, DCT-5C, MP-77, MP-77B and MP-77C:

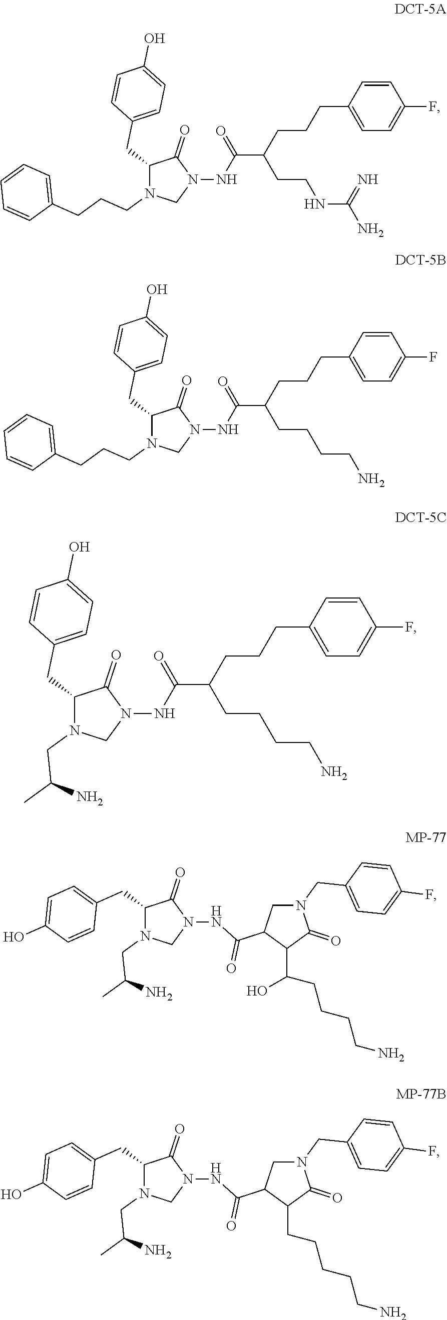 [SMILES: N=C(N)NCCC(CCCc1ccc(F)cc1)C(=O)NN1CN(CCCc2ccccc2)[C@H](Cc2ccc(O)cc2)C1=O ; NCCCCC(CCCc1ccc(F)cc1)C(=O)NN1CN(CCCc2ccccc2)[C@H](Cc2ccc(O)cc2)C1=O ; C[C@H](N)CN1CN(NC(=O)C(CCCCN)CCCc2ccc(F)cc2)C(=O)[C@H]1Cc1ccc(O)cc1 ; C[C@H](N)CN1CN(NC(=O)C2CN(Cc3ccc(F)cc3)C(=O)C2C(O)CCCCN)C(=O)[C@H]1Cc1ccc(O)cc1 ; C[C@H](N)CN1CN(NC(=O)C2CN(Cc3ccc(F)cc3)C(=O)C2CCCCCN)C(=O)[C@H]1Cc1ccc(O)cc1]

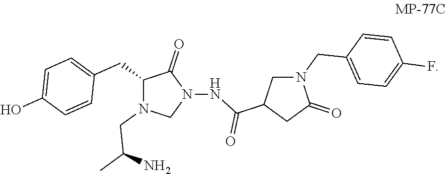 [SMILES: C[C@H](N)CN1CN(NC(=O)C2CC(=O)N(Cc3ccc(F)cc3)C2)C(=O)[C@H]1Cc1ccc(O)cc1]

In one embodiment, the invention relates to a method of inhibiting proteinase 3 (PR3) or Human Neutrophil Elastase (HNE) and Cathespin G (Cat G/CathG) in a patient. The method can include administering an effective amount of one or more of the compounds described herein. Compounds of the invention include compounds DCT-5 (and related compounds) and MP-77 (and related compounds) as described herein.

In one embodiment, compounds described herein are administered to a patient to suppress an undesired neutrophil immune response related to activation of the PR3 receptor.

Embodiments also include methods of treating an ailment. The ailment can be, for example, an inflammatory disorder, inflammation condition, autoimmune or autoinflammatory disease. The methods can include administering a therapeutic amount of a compound selected from DCT-5A, DCT-5B, DCT-5C, MP-77, MP-77B and MP-77C.

In aspects, the ailment is one or more of ANCA-associated vasculitis, ulcerative colitis, systemic lupus, erythematosus, rheumatoid arthritis, gout or IBD.

In aspects, the ailment is one or more of cystic fibrosis, COPD, asthma or transfusion-related acute lung injury.

In aspects, the ailment is one or more of early endothelial dysfunction, atherosclerosis, atherothrombosis or venous thrombosis.

In aspects, the ailment is granulomatosis with polyangiitis (GPA) or another disease/ailment where PR3 ANCA or NETosis is part of disease pathology.

Embodiments also include methods of inhibiting the activity of Proteinase 3 (PR3) and/or Human Neutrophil Elastase (HNE). The methods can include administering a therapeutic amount of one or more of the compounds described herein such as DCT-5A, DCT-5B, DCT-5C, MP-77, MP-77B and MP-77C.

In aspects binding of anti-neutrophil cytoplasmic antibodies (ANCA) to PR3 is inhibited.

In aspects use of a compound described herein replaces the function of alpha-1 protease inhibitor and is utilized inhibiting protease activity of human neutrophil elastase (HNE).

In aspects, the effective amount of a compound described herein is from about 0.01 mg/kg to 10 mg/kg body weight of the patient.

In aspects, a compound described herein features a salt bridge to Asp61.

In aspects, appendages (generally containing basic functions) can reach into S1/S′ pocket or either proteinase 3 (PR3) or human neutrophil elastase (HNE) resulting in additional peptide interactions.

In aspects, small functional groups (e.g., OH, F and NH₂) on appendages and terminal aromatic groups can increase binding within the S1/S′ pocket.

In aspects, the central amide group (H bond with Hys57) is exchanged with imidazole and the imidazolidinone (left ring) can be exchanged with 5- and 6-membered aromatic rings.

Embodiments include a compound, or a pharmaceutically acceptable salt thereof, wherein the compound is selected from JK-58, JK-53, JK-169, JK-178, PEH-126, PEH-109, JK-205, PEH-129, PEH-111, KO-4, JK-187, JK-123, MP-76, JK-78, JK146, JK-145, JK121, JK138, JK-141, JK-76, MP-46, JK-99, JK-87, JK-25, JK-1, PEH-70, DCT-1, DCT-8, DCT-7, MP-48, PEH-73, PEH-101, PEH-100, PEH-98, PEH-81, DCT-10, DCT-3 and DCT-6:

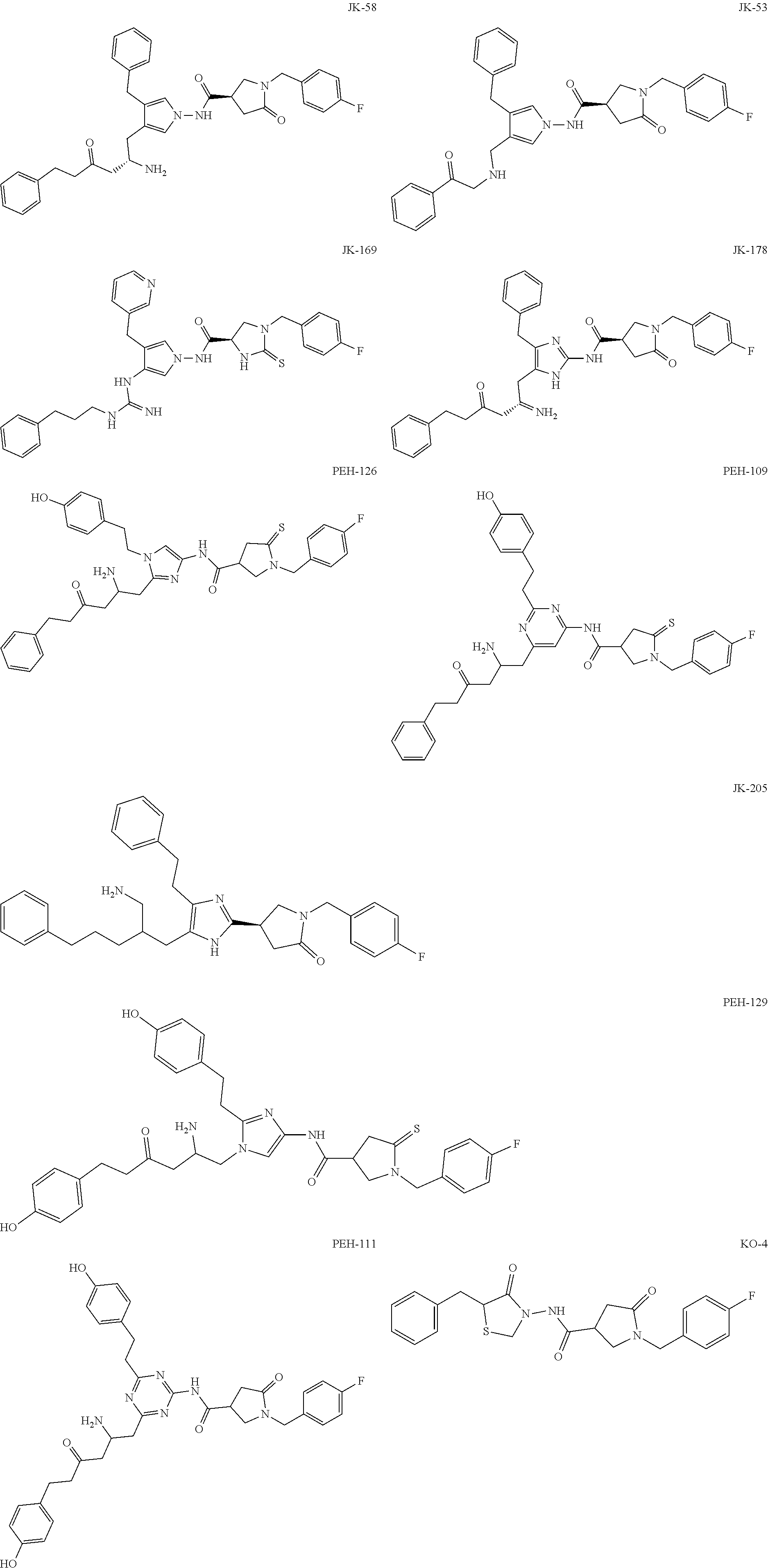

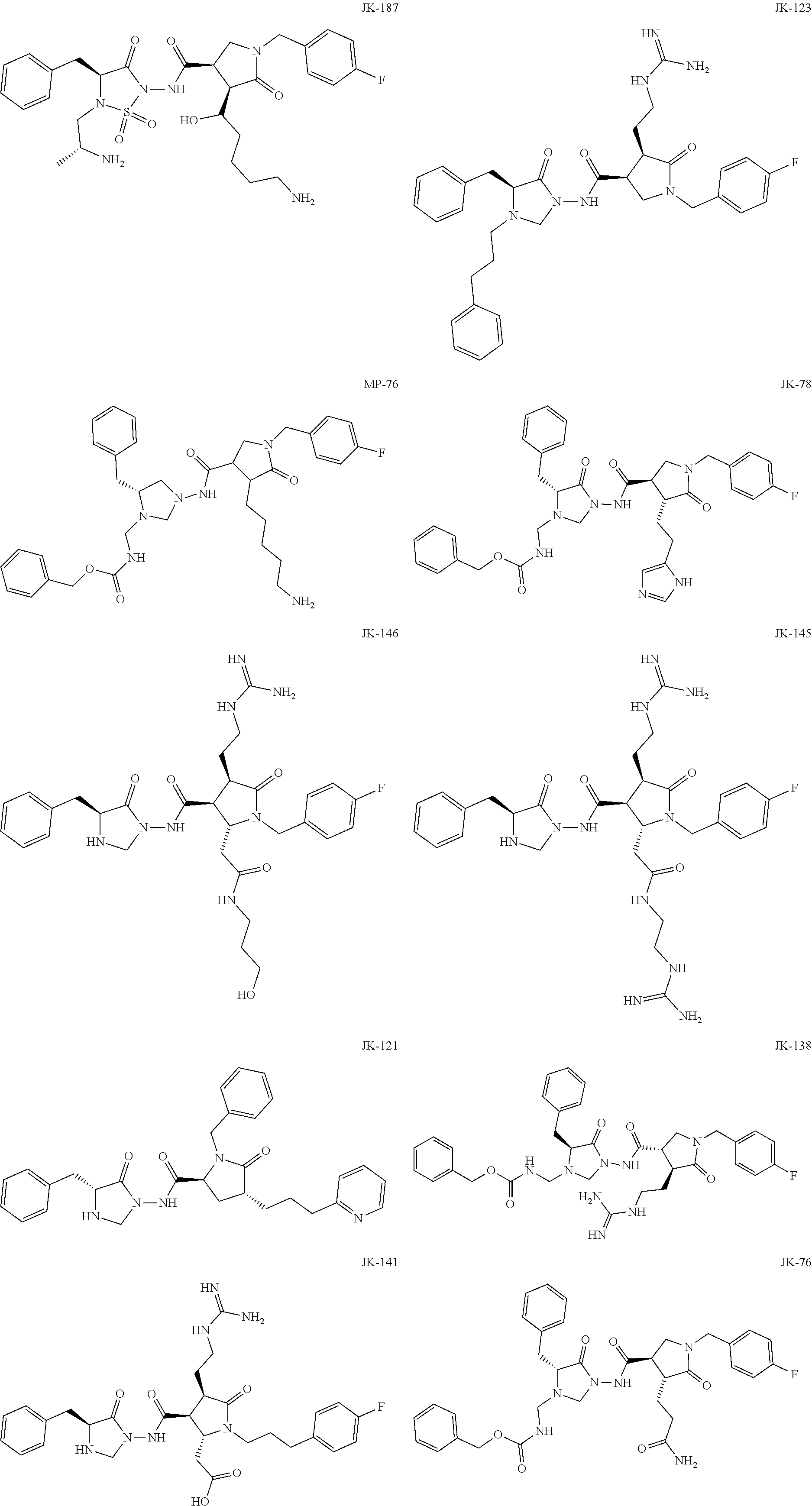

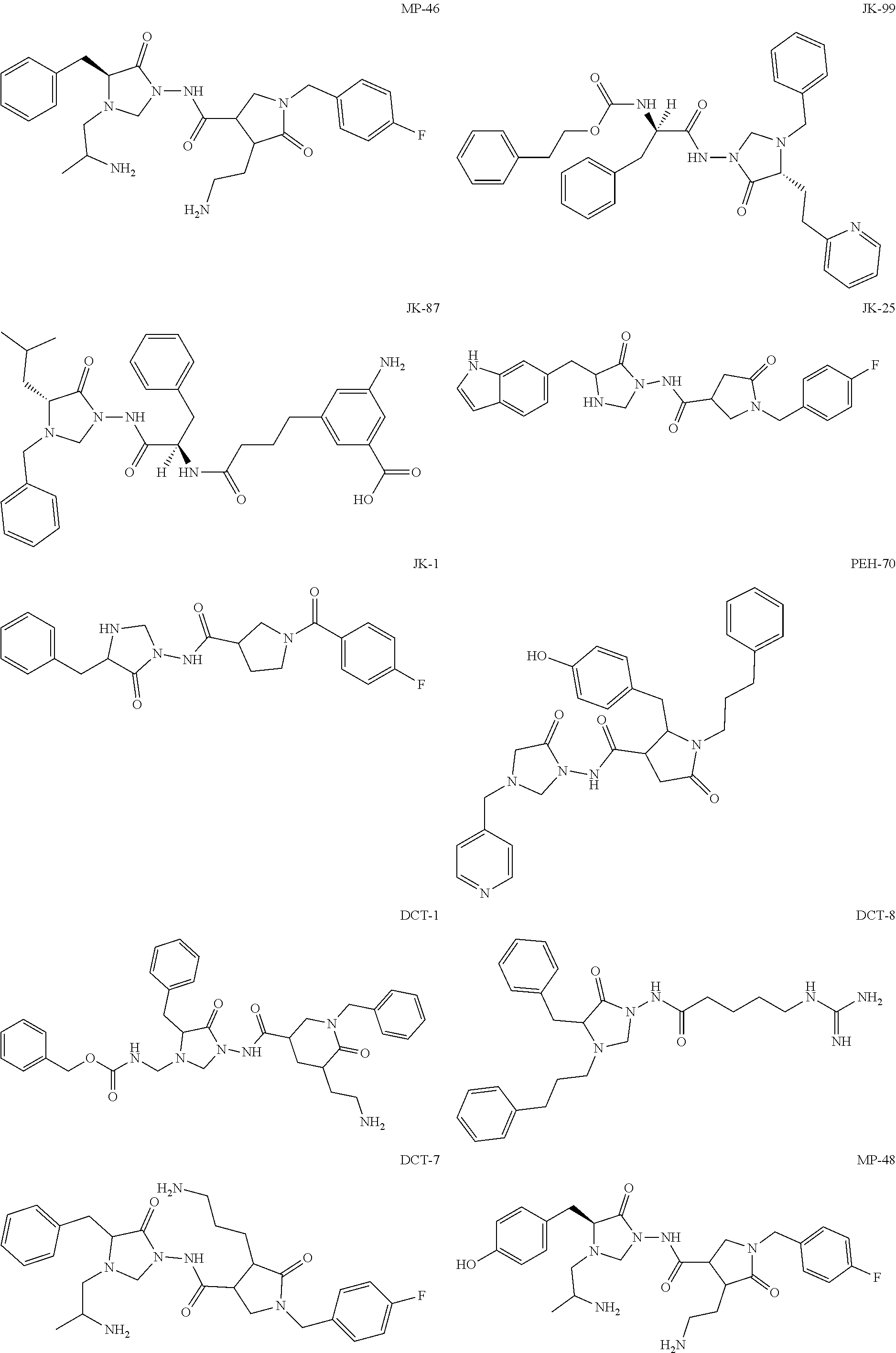

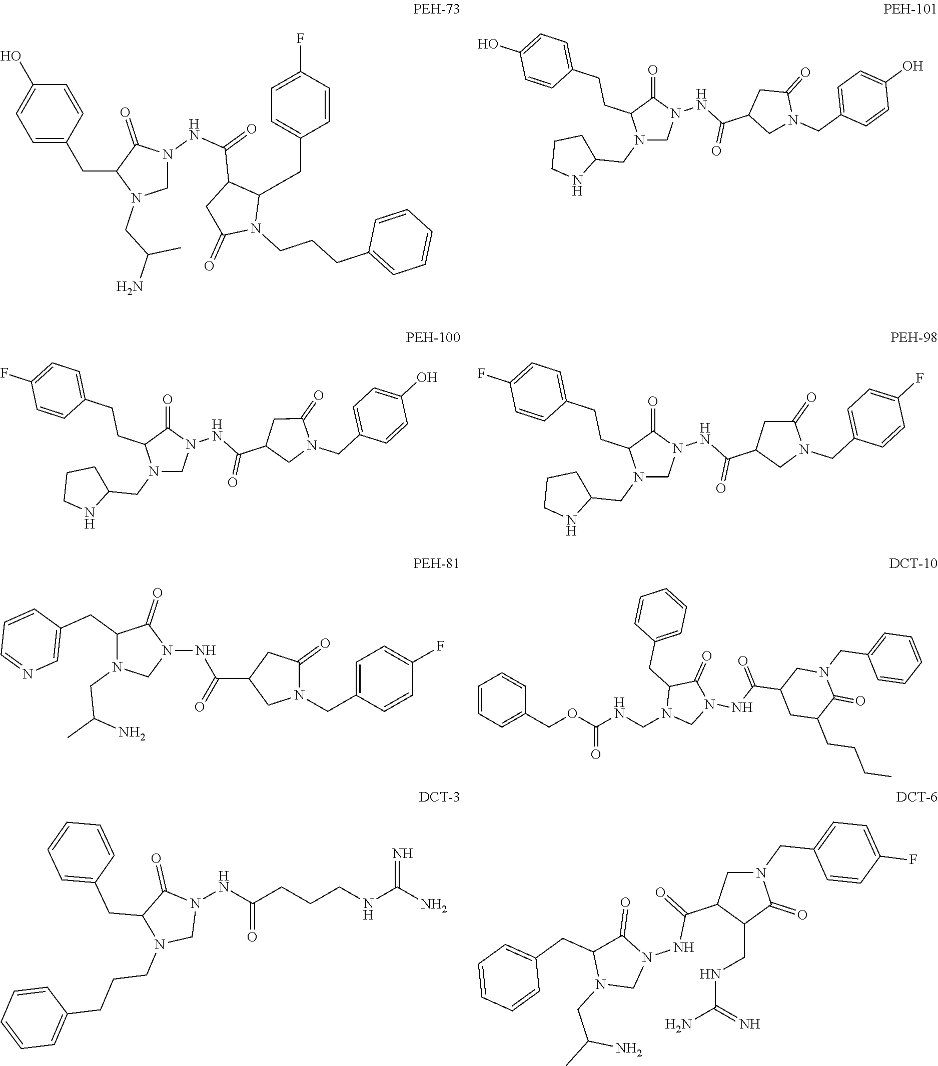

Embodiments also include methods of treating an ailment using one or more of the compounds described herein. Specifically, the methods can include administering a therapeutic amount of JK-58, JK-53, JK-169, JK-178, PEH-126, PEH-109, JK-205, PEH-129, PEH-111, KO-4, JK-187, JK-123, MP-76, JK-78, JK-146, JK-145, JK-121, JK138, JK141, JK-76, MP-46, JK-99, JK-87, JK-25, JK-1, PEH-70, DCT-1, DCT-8, DCT-7, MP-48, PEH-73, PEH-101, PEH-100, PEH-98, PEH-81, DCT-10, DCT-3 and/or DCT-6.

In aspects, the ailment is an inflammatory disorder, inflammation condition, autoimmune or autoinflammatory disease. In aspects, the ailment is one or more of ANCA-associated vasculitis, ulcerative colitis, systemic lupus, erythematosus, rheumatoid arthritis, gout or IBD.

In aspects, the ailment is one or more of cystic fibrosis, COPD, asthma or transfusion-related acute lung injury.

In aspect, the ailment is one or more of early endothelial dysfunction, atherosclerosis, atherothrombosis or venous thrombosis.

In aspects, the ailment is granulomatosis with polyangiitis (GPA) or another disease/ailment where PR3 ANCA or NETosis is part of disease pathology.

Embodiments also include methods of inhibiting the activity of Proteinase 3 (PR3) and/or Human Neutrophil Elastase (HNE). The methods can include administering a therapeutic amount of one or more of the compounds of JK-58, JK-53, JK-169, JK-178, PEH-126, PEH-109, JK-205, PEH-129, PEH-111, KO-4, JK-187, JK-123, MP-76, JK-78, JK146, JK-145, JK-121, JK-138, JK-141, JK-76, MP-46, JK-99, JK-87, JK-25, JK-1, PEH-70, DCT-1, DCT-8, DCT-7, MP-48, PEH-73, PEH-101, PEH-100, PEH-98, PEH-81, DCT-10, DCT-3 and DCT-6.

In aspects binding anti-neutrophil cytoplasmic antibodies (ANCA) is inhibited.

In aspects use of a compound described herein replaces the function of alpha-1 protease inhibitor and is utilized inhibiting protease activity of human neutrophil elastase (HNE).

In aspects, the effective amount of a compound described herein is from about 0.01 mg/kg to 10 mg/kg body weight of the patient.

In aspects, a compound described herein features a salt bridge to Asp61.

In aspects, appendages (generally containing basic functions) can reach into S1/S′ pocket or either proteinase 3 (PR3) or human neutrophil elastase (HNE) resulting in additional peptide interactions.

In aspects, small functional groups (e.g., OH, F and NH₂) on appendages and terminal aromatic groups can increase binding within the S1/S′ pocket.

In aspects, the central amide group (H bond with Hys57) is exchanged with imidazole and the imidazolidinone (left ring) can be exchanged with 5- and 6-membered aromatic rings.

In aspects, a compound described herein is administered along with an immunosuppressive agent. The immunosuppressive agent can be, for example, cyclophosphamide, rituximab, methotrexate, azathioprine or a glucocorticoid.

### BRIEF DESCRIPTION OF THE DRAWINGS

The accompanying drawings illustrate aspects of the present invention. In such drawings:

FIG. 1A is a two-dimensional representation of docked enumerated ligand MP-77 against PR3.

FIG. 1B is a two-dimensional representation of docked enumerated ligand DCT-5 against PR3.

FIG. 1C is a two-dimensional representation of docked enumerated ligand JK-58 against PR3.

FIG. 1D is a surface representation of PR3 binding pocket with docked PEH-109 (dark sticks) and PEH-126 (light sticks).

FIG. 1E is a surface representation of PR3 binding pocket with docked MP-77, DCT-5 and JK-58. Key interactions between compounds and binding pocket residues are highlighted.

FIG. 2A depicts the steps in the chemical synthesis of DCT-5A.

FIG. 2B depicts the steps in the chemical synthesis of DCT-5B and DCT-5C.

FIG. 3A depicts the steps in the chemical synthesis of MP-77.

FIG. 3B depicts the steps in the chemical synthesis of MP-77B and MP-77C.

FIG. 4 depicts molecules MP-77 and MP-77B as drawn with no stereocontrol of the lactam carbons; MP-77X represented as one compound (variable values for R¹, R² and length of chain); DCT-5A and DCT-5B with no stereocontrol at alpha carbon to hydrazide (amide); and DCT-5X represented as one compound (variable value for R² and length of chain).

FIG. 5A is a table of the “Simplified Molecular Input Line Entry System” or SMILES of each compound.

FIG. 5B is a table that lists in silico molecular docking studies and MM/GBSA analysis for each molecule.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “neutrophil” refers to a white blood cell which operates as the first line of defense in an immune system. Other known definitions within the art as applicable to neutrophil are also contemplated throughout this disclosure. Neutrophils are the most abundant form of granulocytes, a type of white blood cell also known as a leukocyte which ranges from about 40-70% of the white blood cell count in the human body.

The term “neutrophil elastase (NE)” refers to a serine proteinase, which is secreted by neutrophils during inflammation. The term “neutrophil elastase (NE)” carries several synonymous monikers, including but not limited to: leukocyte elastase, ELANE, ELA2, elastase 2, neutrophil, elaszym, serine elastase, and subtype human leukocyte elastase (HLE). All of these aforementioned terms are synonymous with ‘neutrophil elastase’ and as such are treated as equivalents within this disclosure and claims to the present invention. Neutrophil elastase (NE) is also known as a serine endopeptidase. Other serine endopeptidases as contemplated in this disclosure include but are not limited to: “proteinase 3 (PR3),” “cathepsin G (CatG/CathG),” and NSP4, all of which contribute to infection and inflammatory processes.

The term “Proteinase 3 (PR3)” refers to an enzyme that is encoded by the human gene PRTN3. Proteinase 3 is a serine protease enzyme expressed mainly in neutrophil granulocytes.

The term “Human Neutrophil Elastase” or “HNE)” refers to a serine proteinase in the same family as chymotrypsin and carries a broad substrate specificity. Human Neutrophil Elastase is secreted by neutrophils during inflammation. Human Neutrophil Elastase (HNE) is synonymous with many monikers, including leukocyte elastase, ELANE, ELA2, elastase 2, neutrophil, elaszym, serine elastase, and subtype human leukocyte elastase (HLE). Other known definitions in the art for the aforementioned terms are inherently contemplated by this disclosure, as well as the other art-accepted terms for the myriad synonyms of Human Neutrophil Elastase (HNE).

The term “neutrophil extracellular traps” or “NETs” refers to net-like structures composed of DNA-histone complexes and proteins released by activated neutrophils. In addition to their key role in the neutrophil innate immune response, NETs are also involved in autoimmune diseases, like systemic lupus erythematosus, rheumatoid arthritis, psoriasis, and in other non-infectious pathological processes, as coagulation disorders, thrombosis, diabetes, atherosclerosis, vasculitis and cancer. Recently, a large body of evidence indicates that NETs are involved in cancer progression and metastatic dissemination, both in animal models and cancer patients. Endogenous deoxyribonuclease I (DNase I) and deoxyribonuclease 1L3 (DNase IL3) enzymes activity is heavily suppressed in diseases accompanied by intensive NETs formation.

A “Neutrophil Extracellular Trap (NET)” is defined as networks of extracellular fibers, primarily composed of DNA from neutrophils, which bind pathogens. Neutrophil Extracellular Traps (NETs) allow neutrophils to kill extracellular pathogens while minimizing damage to the host cells. Neutrophils release granule proteins and chromatin to form an extracellular fibril matrix known as NET through an active process.

“Polymorphonuclear neutrophils” are defined as neutrophils which contain at least four serine endopeptidases. Namely: neutrophil elastase (NE), proteinase 3 (PR3), cathepsin G (CatG/CathG), and NSP4, which contribute to the regulation of infection and inflammatory processes. Polymorphonuclear neutrophils are part of the polymorphonuclear cells family (PMN) together with basophils and eosinophils. Polymorphonuclear neutrophils are also known as polymorphonuclear leukocytes and can be part of what are known as ‘Glitter Cells.’ Glitter cells are polymorphonuclear leukocyte neutrophils accompanied by granules. Other art-recognized definitions and synonyms of polymorphonuclear neutrophils are inherently contemplated by this disclosure.

The term “Cathepsin G,” “CatG” or “CathG” refers to a protein that is encoded by the human GTSG gene. It is a serine protease of the chymotrypsin family and also a member of the peptidase S1 protein family. Cathepsin G acts to eliminate intracellular pathogens and breaks down tissues at inflammatory sites, as well as contributing to anti-inflammatory response. Cathepsin G is a 255-amino-acid-residue protein including an 18-residue signal peptide, a two-residue activation peptide at the N-terminus and a carboxy terminal extension. As a component of neutrophil proteolytic machinery, Cathepsin G regulates the inflammatory responses by stimulating the production of cytokines and chemokines, which are responsible for the activation and mobilization of immune cells to the site of pathogen and/or tissue damage. Cathepsin G is further defined as a serine protease that controls the functional state of immune cells and is traditionally considered to be one of the effectors of inflammation.

The term “NSP4” refers to a transmembrane glycoprotein that is organized into three main domains: a three-helical TM domain in the N-terminus, a central cytoplasmic coiled-coil domain for multimerization, and a C-terminal flexible region. The N-terminus is also known as a viroporin domain. NSP4 can be secreted out of the cell and oligomerizes into dimeric, tetrameric, pentameric and higher-order forms. All art-accepted and art-recognized definitions and synonyms for ‘NSP4’ are inherently contemplated by this disclosure.

The term “Elafin” refers to a polypeptide inhibitor of some peptidases and elastases. It is also known as “peptidase inhibitor 3” or “skin-derived antileukoprotease (SKALP).”

The term “inflammation” refers to part of the complex biological response of body tissues to harmful stimuli, such as pathogens, damaged cells, or irritants, and is a protective response involving immune cells, blood vessels, and molecular mediators. The function of inflammation is to eliminate the initial cause of cell injury, clear out necrotic cells and tissues damaged from the original insult and the inflammatory process, and initiate tissue repair. The five cardinal signs are heat, pain, redness, swelling, and loss of function. Inflammation is a generic response, and therefore it is considered as a mechanism of innate immunity, as compared to adaptive immunity, which is specific for each pathogen. Too little inflammation could lead to progressive tissue destruction by the harmful stimulus (e.g., bacteria) and compromise the survival of the organism. In contrast, too much inflammation, in the form of chronic inflammation, is associated with various diseases, such as hay fever, periodontal disease, atherosclerosis, and osteoarthritis. Inflammation can be classified as either acute or chronic. Acute inflammation is the initial response of the body to harmful stimuli and is achieved by the increased movement of plasma and leukocytes (in particular granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process.

The term “inflammatory disorder” or “inflammation condition” refers to a condition in which the immune system mistakenly attacks one's own cells or tissues. This causes abnormal inflammation that can result in chronic pain, redness, swelling, stiffness and damage to otherwise healthy body tissues. Inflammatory conditions can affect the nervous system (e.g., encephalitis, myelitis, meningitis, arachnoiditis and neuritis). Inflammatory conditions can affect the eyes (e.g., dacryoadenitis, scleritis, episcleritis, keratitis, retinitis, chorioretinitis, blepharitis, conjunctivitis and uveitis). Inflammatory conditions can affect the ears (e.g., Otitis externa, Otitis media, Labyrinthitis and Mastoiditis). Inflammatory conditions can affect the cardiovascular system (e.g., Endocarditis, Myocarditis, Pericarditis, Arteritis, Phlebitis and Capillaritis). Inflammatory conditions can affect the respiratory system (e.g., Sinusitis, Rhinitis, Pharyngitis, Laryngitis, Tracheitis, Bronchitis, Bronchiolitis, Pneumonitis, Pleuritis and Mediastinitis. Inflammatory conditions can affect the mouth and digestive system (e.g., Stomatitis, Gingivitis, Gingivostomatitis, Glossitis, Tonsillitis, Sialadenitis/Parotitis, Cheilitis, Pulpitis, Gnathitis, Esophagitis, Gastritis, Gastroenteritis, Enteritis, Colitis, Enterocolitis, Duodenitis, Ileitis, Caecitis, Appendicitis and Proctitis). Inflammatory conditions can affect the accessory digestive organs (e.g., Hepatitis, Ascending cholangitis, Cholecystitis, Pancreatitis and Peritonitis). Inflammatory conditions can affect the integumentary system (e.g., Dermatitis, Folliculitis, Cellulitis and Hidradenitis). Inflammatory conditions can affect the musculoskeletal system (e.g., Arthritis, Dermatomyositis, Myositis, Synovitis/Tenosynovitis, Bursitis, Enthesitis, Fasciitis, Capsulitis, Epicondylitis, Tendinitis, Panniculitis, Osteochondritis: Osteitis/Osteomyelitis, Spondylitis, Periostitis and Chondritis). Inflammatory conditions can affect the urinary system (e.g., Nephritis, Glomerulonephritis, Pyelonephritis, Urethritis, Cystitis and Urethritis). Inflammatory conditions can affect the female reproductive system (e.g., Oophoritis, Salpingitis, Endometritis, Parametritis, Cervicitis, Vaginitis, Vulvitis and Mastitis). Inflammatory conditions can affect the male reproductive system (e.g., Orchitis, Epididymitis, Prostatitis, Seminal vesiculitis, Balanitis, Posthitis and Balanoposthitis. Inflammatory conditions can affect the endocrine system (e.g., Insulitis, Hypophysitis, Thyroiditis, Parathyroiditis and Adrenalitis). Inflammatory conditions can also affect the lymphatic system (e.g., Lymphangitis and Lymphadenitis).

The term “autoimmune disease” or “autoimmune disorder” refers to a condition arising from an abnormal immune response to a functioning body part. Common autoimmune diseases include Addison disease, Celiac disease, Dermatomyositis, Graves disease, Hashimoto thyroiditis, Multiple sclerosis, Myasthenia gravis and Pernicious anemia.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

A “pharmaceutical composition” can include the combination of an active agent, such as a therapeutic molecule, with a carrier, inert or active, in a sterile composition suitable for diagnostic or therapeutic use in vitro, in vivo or ex vivo.

As used herein, the term “prevention” means all of the actions by which the occurrence of the disease is restrained, ameliorated or retarded.

The term “treating” or “treatment” refers to one or more of (1) inhibiting the disease (e.g., inhibiting a disease, condition or disorder) in an individual who is experiencing or displaying the pathology or symptomatology of the disease, condition or disorder (i.e., arresting further development of the pathology and/or symptomatology); and (2) ameliorating the disease (e.g., ameliorating a disease, condition or disorder) in an individual who is experiencing or displaying the pathology or symptomatology of the disease, condition or disorder (i.e., reversing the pathology and/or symptomatology) such as decreasing the severity of disease.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration.

The term “subject” or “patient” refers to those intended to be treated with an active agent. Optionally, the subject is someone who is susceptible to inflammatory diseases and inflammatory conditions. However, any subject to be treated with the pharmaceutical composition and compounds disclosed herein is included without limitation. In certain embodiments, the pharmaceutical composition disclosed herein is administered to a subject to prevent and/or treat inflammatory diseases and disorders.

The term “ion pair” refers to a natural species formed in solution by electrostatic interaction between oppositely charged states that are often sufficiently lipophilic to dissolve in a non-aqueous solvent.

The term “ion pairing” refers to an approach to improve the lipophilicity and subsequently the transport rate of a drug across lipid membranes. Lipophilization (esterification of a lipophilic moiety) of ionic drugs with high aqueous solubilities by ion-pair formation with appropriate counter-ion have proven to be successful in this regard. An ion-pair is a pair of oppositely charged ions held together by Coulombic attraction without formation of a covalent bond. They can behave like a single unit and partition into the membrane as a more lipophilic unit. This strategy involves co-administering an excess concentration of a counter-ion.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The therapeutic agents in the pharmaceutical compositions and compounds can be formulated in a “therapeutically effective amount” or a “prophylactically effective amount.” A “therapeutically effective amount” refers to an amount effective, at dosages and for periods of time necessary, to achieve the desired therapeutic result. A therapeutically effective amount of the recombinant vector may vary depending on the condition to be treated, the severity and course of the condition, the mode of administration, whether the peptide or agent is administered for preventive or therapeutic purposes, the bioavailability of the particular agent(s), the ability of the agent to elicit a desired response in the individual, previous therapy, the age, weight and sex of the patient, the patient's clinical history and response to the agent, the type of the therapeutic agent used, discretion of the attending physician, etc. A therapeutically effective amount is also one in which any toxic or detrimental effects of the recombinant vector are outweighed by the therapeutically beneficial effects. A “prophylactically effective amount” refers to an amount effective, at dosages and for periods of time necessary, to achieve the desired prophylactic result.

Many known and useful compounds and the like can be found in Remington's Pharmaceutical Sciences (13th Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include

“In silico modeling” refers to experiments performed on a computer, or on any device containing computer chips, processor chips, semiconductors, a central processing unit (CPU), or any device which performs computer simulations, simulations, or produces models or performs modelling.

An “S1 pocket” generally refers to a negatively charged aspartic acid or glutamic acid present at the base of an enzyme. Furthermore, enzymes possess myriad other pockets within their structure, these pockets can be generally referred to as “S pockets.” S pockets, S1 pockets, and all other art-recognized equivalents are contemplated by this disclosure.

“Schrödinger Suite” refers to a collection of software with myriad applications associated with computational modeling. This suite of software applications provides myriad functions, tools, and services, including but not limited to: comprehensive protein modeling, homology modeling tools, chimeric modeling and models, multimeric modeling and models, sequencing tools, protein structure quality analysis tools, identification of structural families and homologs tools, protein engineering, protein aggregation prediction, identification of hot spots for proteolysis, glycosylation, deamidation, and oxidation. Further, the Schrödinger Suite provides tools, software, and support for antibody modeling, prediction of CDR from sequence, rapid prediction utilizing a curated antibody database, advanced ab initio loop prediction, and database management tools for simple incorporation of new and proprietary structures, allowing the use of multiple databases in modeling. Additional functionality of Schrödinger Suite further encompasses: protein docking, utilizing docking code such as PIPER, special antibody and multimer modes of modeling, advanced simulations, advanced molecular dynamics (MD), extensive free energy perturbation (FEP) tools, quantum mechanics/molecular mechanics (QM/MM) predictions of binding site reactivity, small-molecule drug discovery modeling, shape-based screening (with or without atom properties), ligand-based pharmacophore modeling, e-Pharmacophore modeling incorporating ligand-receptor interaction energies, flexible ligand docking with industry-leading Glide, SIFt (structure interaction fingerprint analysis), induced-fit docking with receptor flexibility, covalent docking, 2D ligand interaction diagrams, advanced computations to estimate binding affinity and to rank-order compounds, post-docking refinement capabilities, Prime MM/GBSA, linear interaction approximation (LIA), QM-polarized ligand docking, analyses to predict, prepare refine, and characterize target structure and binding modes, protein crystal structure refinement, protein structure analysis, homology modeling, GPCR modeling, hERG modeling, protein binding site identification, protein binding site analysis, multiple binding mode prediction, utilities to prepare, analyze and filter ligand structures and to create and design ligand libraries, 2D to 3D structure conversion, tautomeric state enumeration, tautomeric state analysis, ligand interaction diagramming, access to commercially available compound databases, flexible ligand superposition, combinatorial library creation, core hopping, filter compound libraries based on predicted ADME properties, R-group analysis, high-performance QM calculations in gas phase and in solution, MM/MD simulations with implicit and explicit solvents, small molecule and macromolecular conformational analyses, mixed-mode QM/MM calculations for ground state and reactivity studies, visualization and workflow automation tools, unified graphical user interface (Maestro) that serves all computations, publication-quality graphics and flexible analysis, KNIME extensions, customizable workflows, and Python API.

“ADME” generally refers to the pharmacometric and pharmacokinetic principle of “Absorption, Distribution, Metabolism, and Excretion” used in pharmacology and in pharmacological and drug modeling. The evaluation of these properties contributes to a conclusion on drug efficacy and describes the disposition of a pharmaceutical compound within an organism. Other related principles applied in this manner are referred to as LADME (Liberation, Absorption, Distribution, Metabolism and Excretion) or ADMET (Absorption, Distribution, Metabolism, Excretion and Toxicity) or LADMET (Liberation, Absorption, Distribution, Metabolism, Excretion and Toxicity).

A “salt bridge” is a combination of two non-covalent interactions: hydrogen bonding and ionic bonding. Ion pairing is one of the most important noncovalent forces in chemistry, in biological systems, in different materials and in many applications such as ion pair chromatography. It is a most commonly observed contribution to the stability to the entropically unfavorable folded conformation of proteins. Although non-covalent interactions are known to be relatively weak interactions, small stabilizing interactions can add up to make an important contribution to the overall stability of a conformer. Not only are salt bridges found in proteins, but they can also be found in supramolecular chemistry. The thermodynamics of each are explored through experimental procedures to access the free energy contribution of the salt bridge to the overall free energy of the state.

Other technical terms used herein convey their ordinary meaning in the art for which they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

An unwanted immune response can occur in autoimmune disease, transplant rejection and allergy. Embodiments include pharmaceutical agents (e.g., neutrophil serine proteases) and methods of suppressing an undesired immune response. A broad NSP inhibitor of HNE, PR3 and Cathepsin G (CathG) can be beneficial for many therapeutic applications.

Embodiments include small molecule inhibitors of PR3 and HNE that were identified using in silico modeling based on binding to what is considered the large and shallow binding pocket for these aforementioned targets. Docking scores, predicted biological and physical properties were calculated for all the proposed compounds. Hydrogen bond interaction to His57 of S pocket to a central amide group of compounds and pi-pi stacking interaction between Phe192 of S1 pocket and terminal phenyl tail of the compounds were predicted to be key interactions to binding both PR3 and HNE as seen for compounds MP-77, DCT-5 and JK-58.

Based on docking results, an increased number of electrostatic interactions predict higher selectivity for compounds toward PR3 (for all three MP-77, DCT-5, and JK-58 compounds). Also, preserving salt bridge and hydrogen bond interactions to Asp61, Lys99 and Trp218 respectively are important for the inhibitor to bind PR3 (since HNE residues 99 and 218 are hydrophobic).

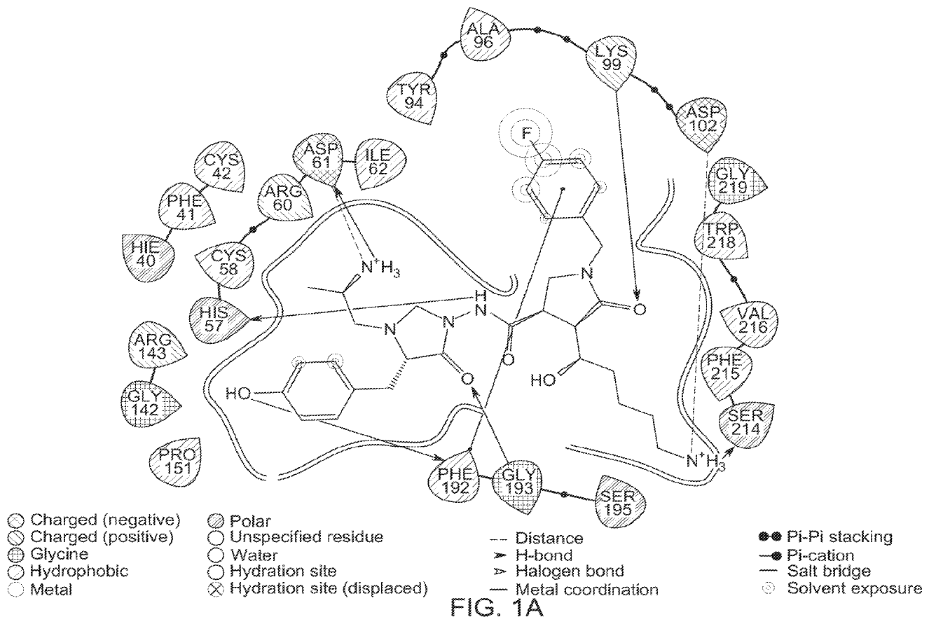

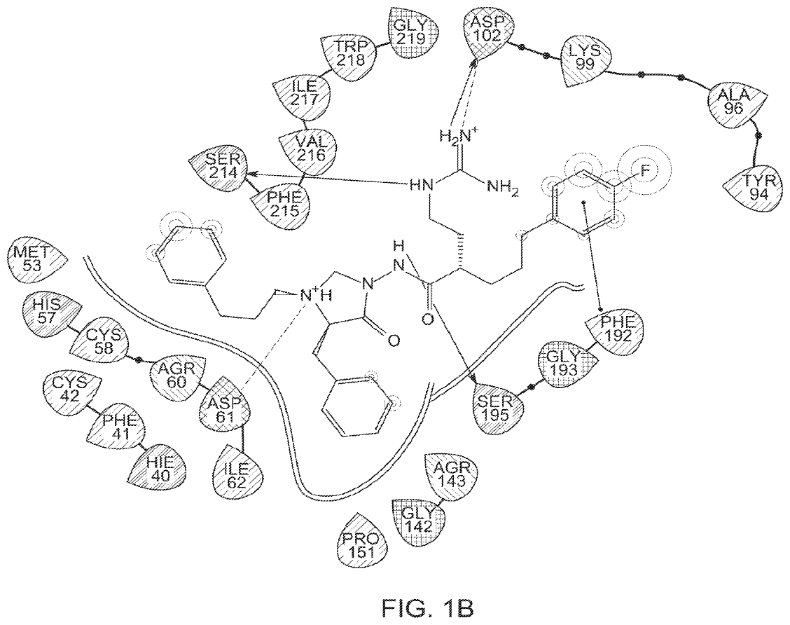

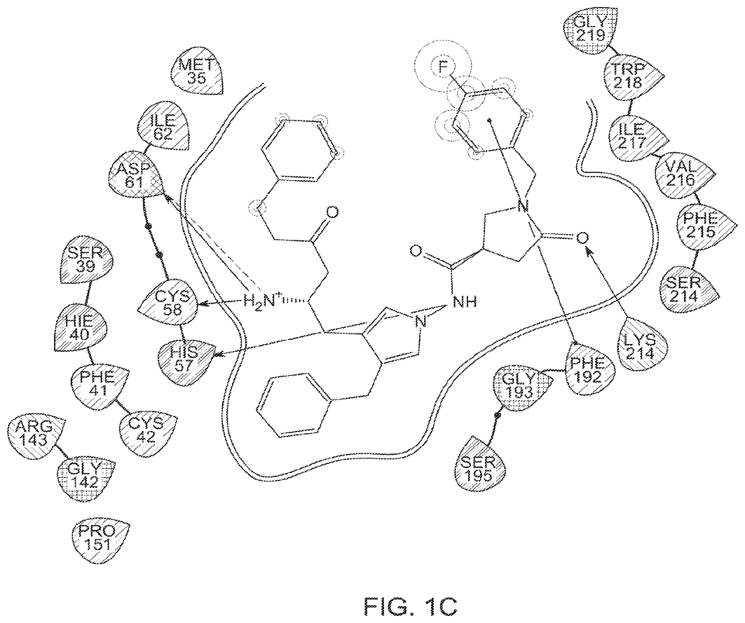

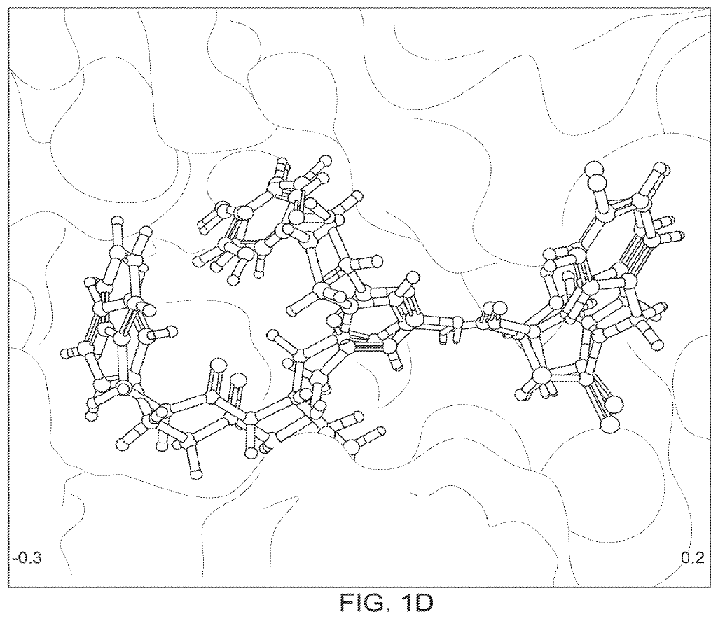

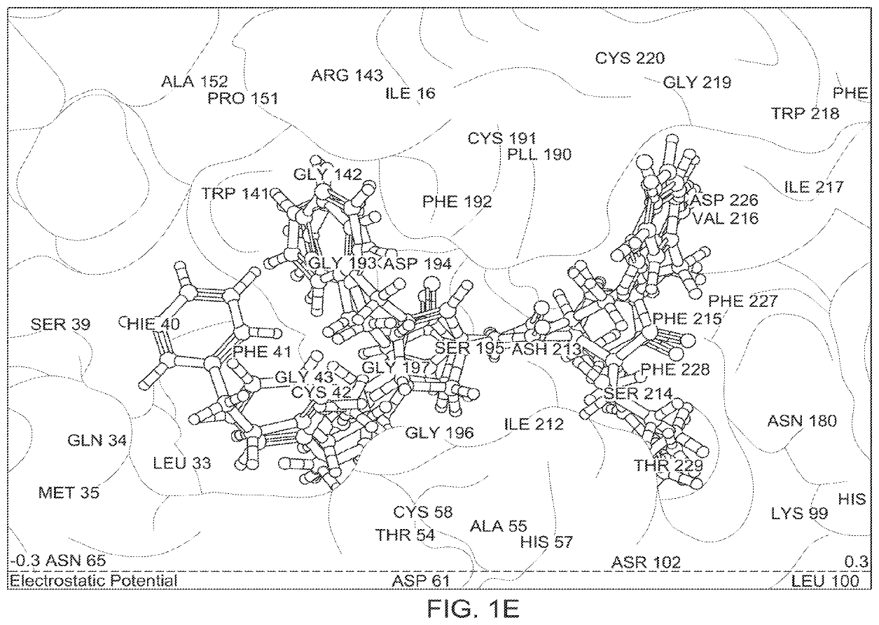

FIG. 1A is a two-dimensional representation of docked enumerated ligand MP-77 against PR3. Similarly, FIG. 1B is a two-dimensional representation of docked enumerated ligand DCT-5 against PR3. FIG. 1C is a two-dimensional representation of docked enumerated ligand JK-58 against PR3. FIG. 1D is a surface representation of PR3 binding pocket with docked PEH-109 and PEH-126.

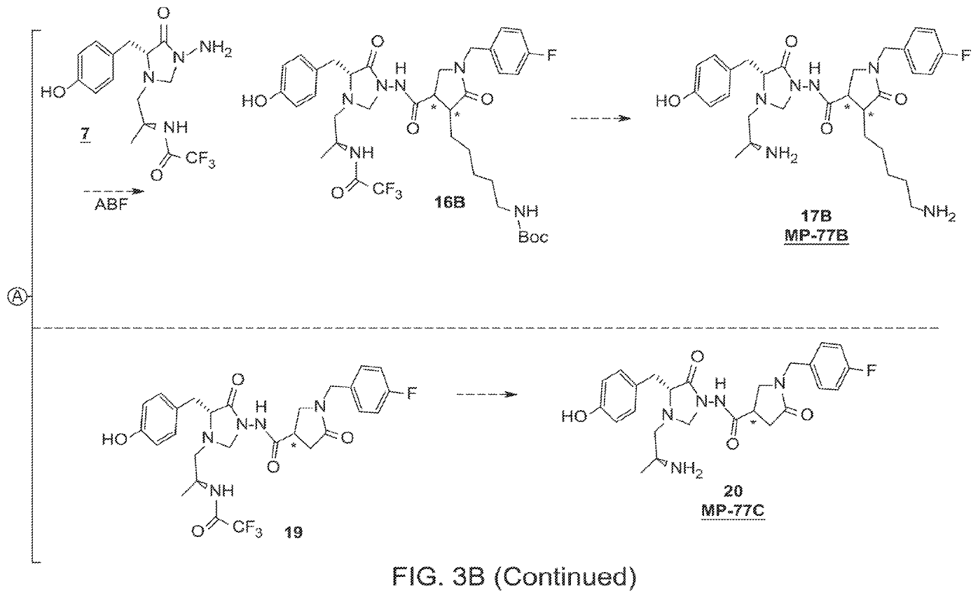

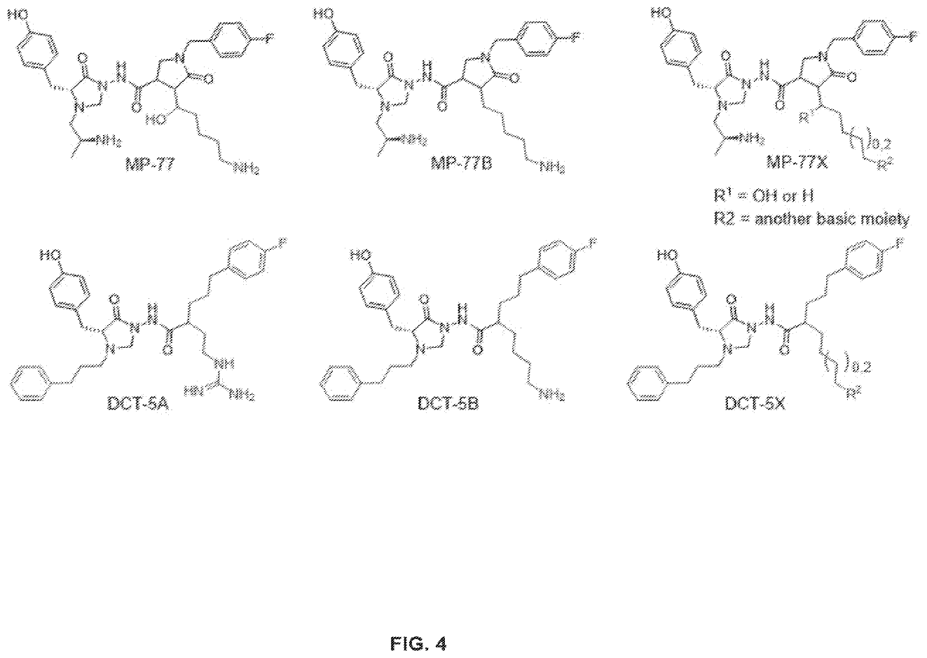

FIG. 1E is a surface representation of PR3 binding pocket with docked MP-77, DCT-5, and JK-58 in green, gray and pink sticks respectively. Key interactions between compounds and binding pocket residues are highlighted. DCT-5 and MP-77 were selected as top hits for synthesis with priority of DCT-5 due to the lowest number of chiral centers (FIG. 4). DCT-5 scaffold provides an additional series arising from the common core imidazolidinone.

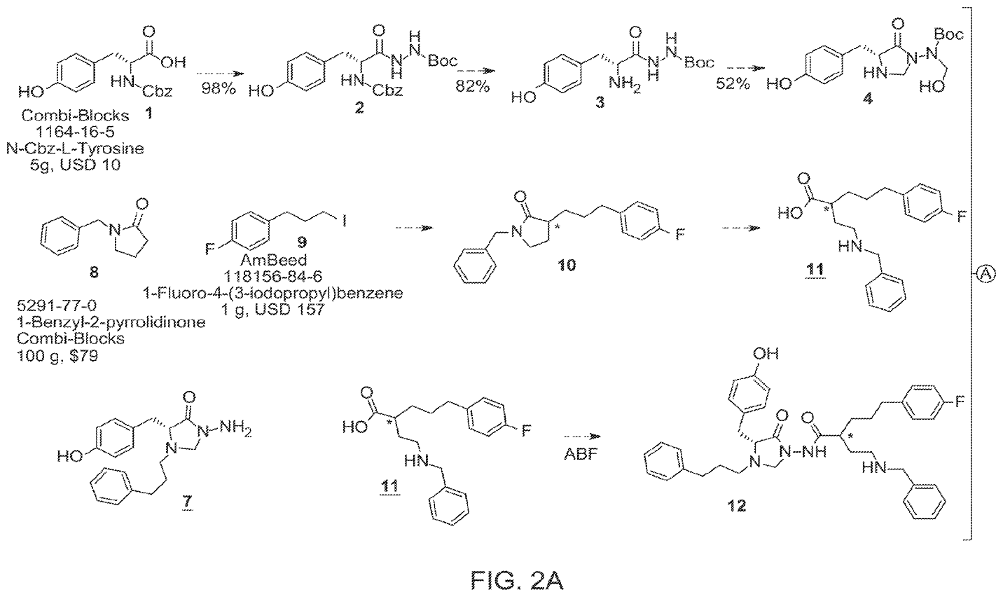

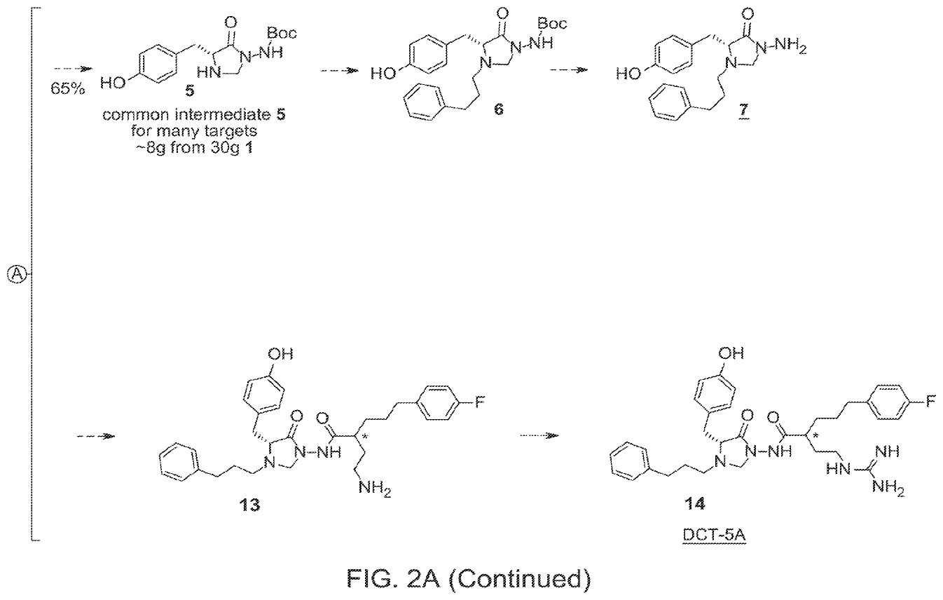

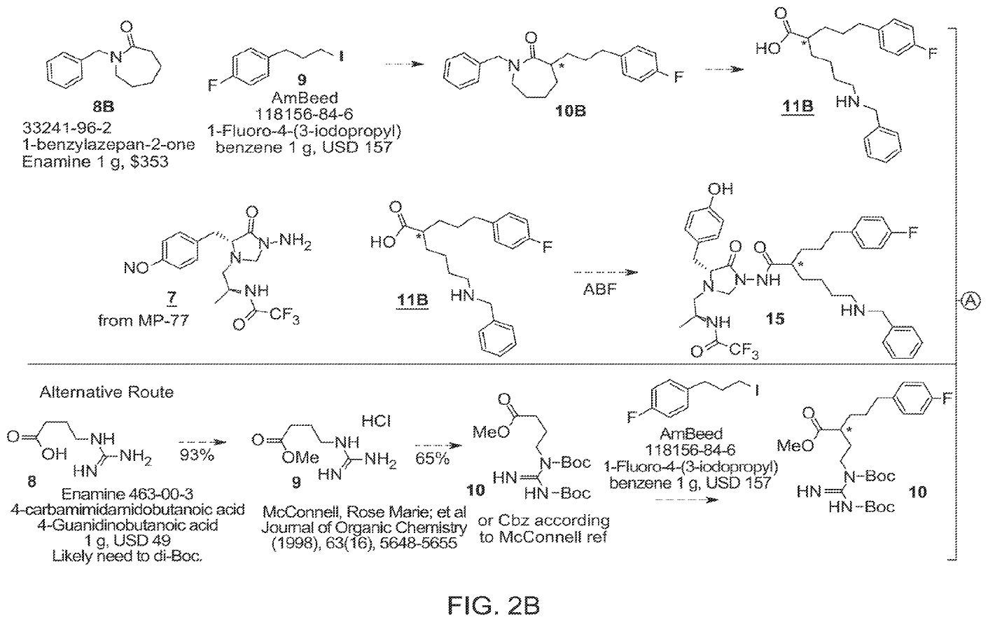

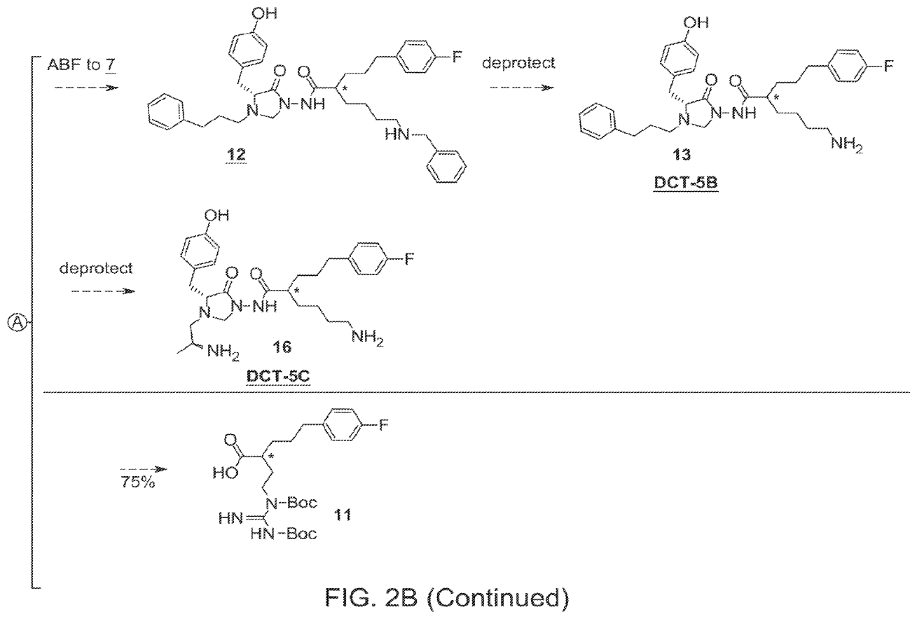

DCT-5A is 5-(4-fluorophenyl)-2-(2-guanidinoethyl)-2-(guanidinoethyl)-N—((R)-4-(4-hydroxybenzyl)-5-oxo-3-(3-phenylpropyl)-imidazolidin-1-(yl)-pentanamide. FIG. 2A depicts the steps in the chemical synthesis of DCT-5A.

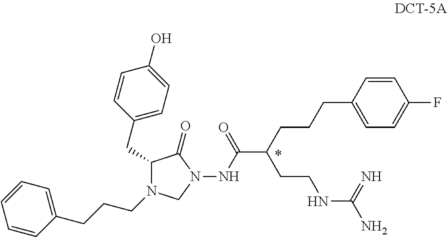 [SMILES: N=C(N)NCCC(CCCc1ccc(F)cc1)C(=O)NN1CN(CCCc2ccccc2)[C@H](Cc2ccc(O)cc2)C1=O]

DCT-5B is 6-amino-2-(3-(4-fluorophenyl)-propyl)-N—((R)-4-(4-hydroxybenzyl)-5-oxo-3-(3-phenylpropyl)-imidazolidin-1-yl)-hexanamide. FIG. 2B depicts the steps in the chemical synthesis of DCT-5B and DCT-5C.

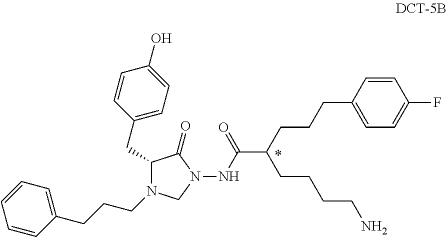 [SMILES: NCCCCC(CCCc1ccc(F)cc1)C(=O)NN1CN(CCCc2ccccc2)[C@H](Cc2ccc(O)cc2)C1=O]

DCT-5C is 6-amino-N—((R)-3-((S)-2-aminopropyl)-4-(4-hydroxybenzyl)-5-oxoimidazolidin-1-yl)-2-(3-(4-fluorophenyl)-propyl)-hexanamide.

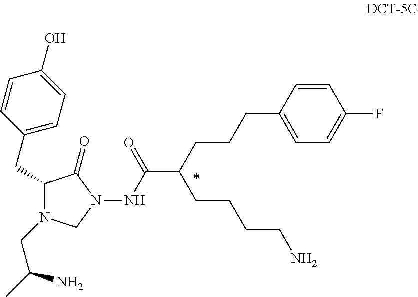 [SMILES: C[C@H](N)CN1CN(NC(=O)C(CCCCN)CCCc2ccc(F)cc2)C(=O)[C@H]1Cc1ccc(O)cc1]

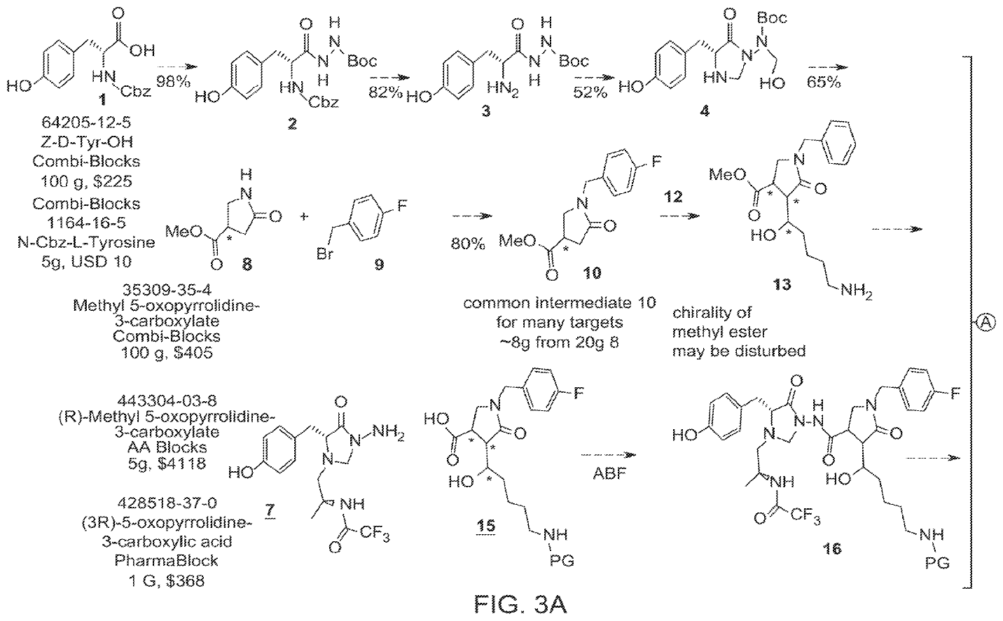

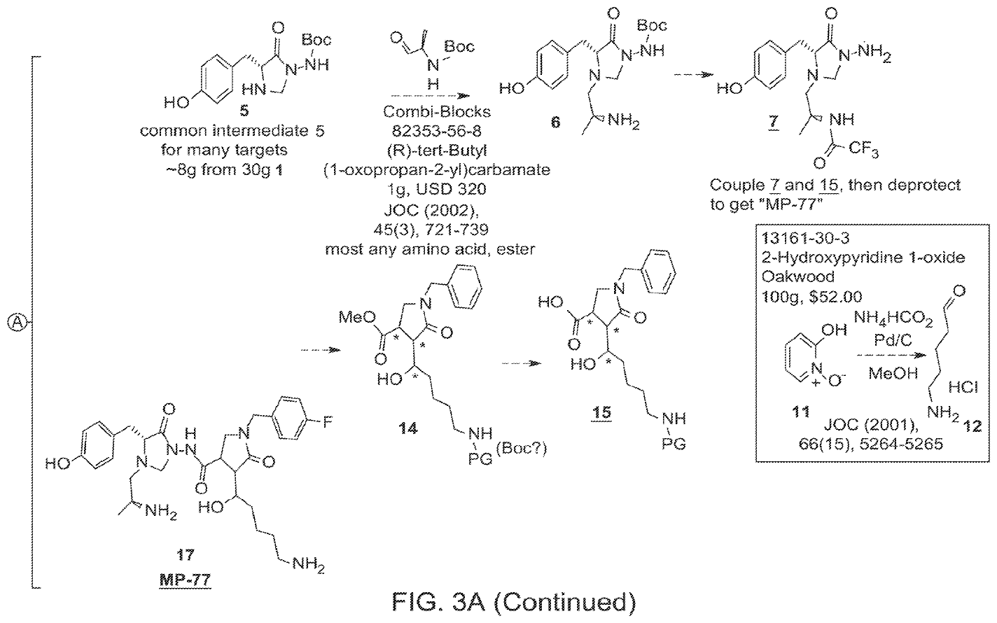

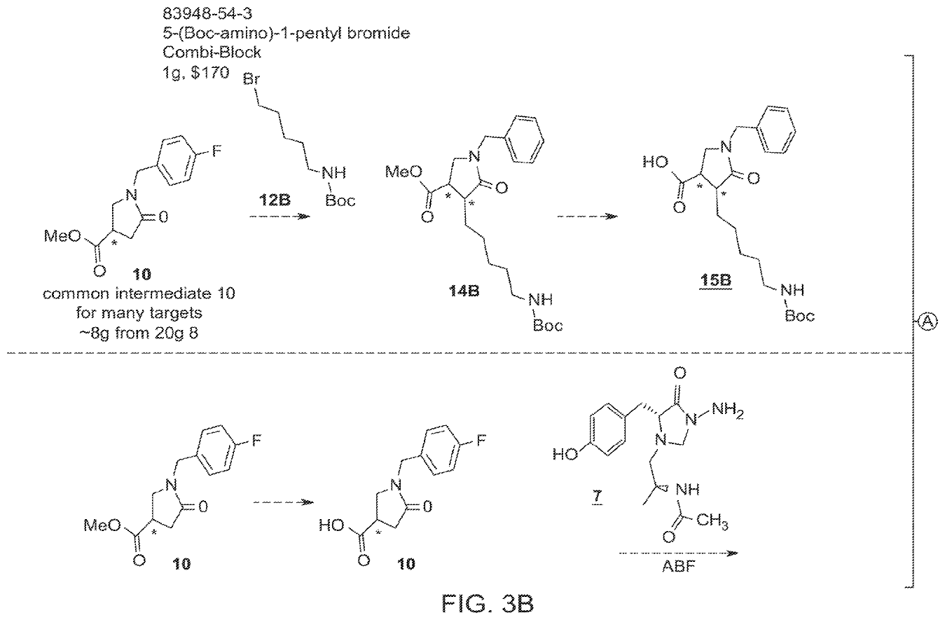

MP-77 is 4-(5-amino-1-hydroxypentyl)-N—((R)-3-((S)-2-aminopropyl)-4-(4-hydroxybenzyl)-5-oxoimidazolidin-1-yl)-1-(4-fluorobenzyl)-5-oxopyrrolidine-3-carboxamide. FIG. 3A depicts the steps in the chemical synthesis of MP-77.

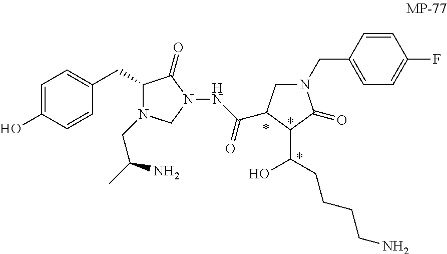 [SMILES: C[C@H](N)CN1CN(NC(=O)C2CN(Cc3ccc(F)cc3)C(=O)C2C(O)CCCCN)C(=O)[C@H]1Cc1ccc(O)cc1]

MP-77B is 4-(5-aminopentyl)-N—((R)-3-((S)-2-aminopropyl)-4-(4-hydroxybenzyl)-5-oxoimidazolidin-1-yl)-1-(4-fluorobenzyl)-5-oxopyrrolidine-3-carboxamide. FIG. 3B depicts the steps in the chemical synthesis of MP-77B and MP-77C.

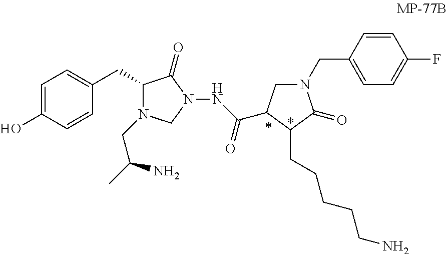 [SMILES: C[C@H](N)CN1CN(NC(=O)C2CN(Cc3ccc(F)cc3)C(=O)C2CCCCCN)C(=O)[C@H]1Cc1ccc(O)cc1]

MP-77C is N—((R)-3-((S)-2-aminopropyl)-4-(4-hydroxybenzyl)-5-oxoimidazolidin-1-yl)-1-(4-fluorobenzyl)-5-oxopyrrolidine-3-carboxamide.

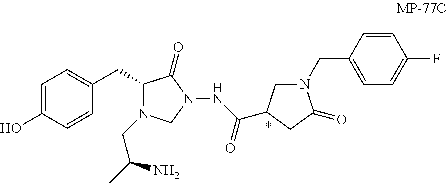 [SMILES: C[C@H](N)CN1CN(NC(=O)C2CC(=O)N(Cc3ccc(F)cc3)C2)C(=O)[C@H]1Cc1ccc(O)cc1]

DCT-5, MP-77 and the other candidate molecules are all non-covalent inhibitors of PR3 that are designed to occupy the S′ subsite of the enzyme. Hydrolysis of a peptide bond by a protease involves binding of a peptide or protein substrate to the active site of the protease. The active site of a protease contains catalytic and binding sites. It includes of an array of pockets or clefts with different shapes surrounded by side chains at the surface of the enzyme that are responsible for substrate specificity.

The listed molecules are reversible inhibitors. As such, they do not form covalent bonds with enzymes. Instead, recognition elements incorporated in the inhibitor allow it to bind to the active site, preventing the approach of a substrate to the enzyme. The ring structure of the listed molecules binds to the S1 pocket of PR3, defined by Ile190 and Phe192. The N—H of the secondary amine donates an H-bond to the carbonyl group of the backbone Ser214.

In one embodiment, the invention provides methods of inhibiting the activity of human neutrophil elastase (HNE) in a patient to treat an ailment. Such methods may comprise administering to the patient an effective amount of DCT-5 (and related compounds) and/or MP-77 (and related compounds). A patient may be of the Alpha-One Antitrypsin Deficient (AATD) genotype or phenotype.

In one embodiment, the present invention provides materials and methods for treating an autoimmune disease in a patient. Such methods may include administering to the patient an effective amount of DCT-5 (and related compounds) and/or MP-77 (and related compounds). Any autoimmune disease may be treated using the compounds of the invention. Examples of autoimmune diseases that can be treated using the compounds of the invention include but are not limited to Granulomatosis with Polyangiitis.

In one embodiment, the invention provides materials and methods of treating a patient with an alternative to the plasma therapeutic alpha-1 antitrypsin (AAT). Such methods may comprise administering an effective amount of a compound of the invention, for example, DCT-5 (and related compounds) and/or MP-77 (and related compounds).

In silico docking studies on newly enumerated compounds against human PR3 structure using high-performance ligand-receptor docking were performed with Glide (Schrödinger Suite). Followed by rank-ordering compounds via Molecular Mechanics-Generalized Born Surface Area (MM-GBSA) calculations to estimate relative binding affinity of hit compounds and provide better enrichment. Since MM-GBSA binding energies (MM-GBSA dG Bind) are approximate free energies of binding, a more negative value indicates stronger binding.

### Administration and Dosing

A pharmaceutical composition comprising a therapeutic molecule in accordance with the present disclosure can be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions can include suitable solid or gel phase carriers or excipients. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases it will be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf-life or effectiveness of the therapeutic agents.

The therapeutic agents in the pharmaceutical compositions can be formulated in a “therapeutically effective amount” or a “prophylactically effective amount.” A “therapeutically effective amount” refers to an amount effective, at dosages and for periods of time necessary, to achieve the desired therapeutic result. A therapeutically effective amount may vary depending on the condition to be treated, the severity and course of the condition, the mode of administration, whether the agent is administered for preventive or therapeutic purposes, the bioavailability of the particular agent(s), the ability of the therapeutic small molecule to elicit a desired response in the individual, previous therapy, the age, weight and sex of the patient, the patient's clinical history and response to the agent, the type of the therapeutic small molecule used, discretion of the attending physician, etc. A therapeutically effective amount is also one in which any toxic or detrimental effects is outweighed by the therapeutically beneficial effects. A “prophylactically effective amount” refers to an amount effective, at dosages and for periods of time necessary, to achieve the desired prophylactic result.

In aspects, an effective amount of the proposed compounds of the present invention or other drug or prodrug product is administered to a subject or a patient in the range of from about 1 ng/kg body weight to about 10 mg/kg body weight, about 1 ng/kg body weight to about 1 mg/kg body weight, about 1 ng/kg body weight to about 100 g/kg body weight, about 1 ng/kg body weight to about 10 g/kg body weight, about 1 ng/kg body weight/day to about 1 g/kg body weight, about 1 ng/kg body weight to about 100 ng/kg body weight, about 1 ng/kg body weight to about 10 ng/kg body weight, about 10 ng/kg body weight to about 100 mg/kg body weight, about 10 ng/kg body weight to about 10 mg/kg body weight, about 10 ng/kg body weight to about 1 mg/kg body weight, about 10 ng/kg body weight/to about 100 g/kg body weight, about 10 ng/kg body weight to about 10 mg/kg body weight, about 10 ng/kg body weight to about 1 mg/kg body weight, 10 ng/kg body weight to about 100 ng/kg body weight, about 100 ng/kg body weight to about 100 mg/kg body weight, about 100 ng/kg body weight to about 10 mg/kg body weight, about 100 ng/kg body weight to about 1 mg/kg body weight, about 100 ng/kg body weight to about 100 mg/kg body weight, about 100 ng/kg body weight to about 10 mg/kg body weight, about 100 ng/kg body weight to about 1 mg/kg body weight, about 1 mg/kg body weight to about 10 mg/kg body weight, about 1 mg/kg body weight to about 100 mg/kg body weight, about 1 mg/kg body weight/day to about 5 mg/kg body weight/day, about 1 mg/kg body weight to about 10 mg/kg body weight, about 1 mg/kg body weight/day to about 100 mg/kg body weight/day, about 5 mg/kg body weight/day to about 10 mg/kg body weight/day, about 10 mg/kg body weight/day to about 50 mg/kg body weight/day, about 10 mg/kg body weight/day to about 100 mg/kg body weight/day, about 10 mg/kg body weight/day to about 1 g/kg body weight/day, about 1 mg/kg body weight to about 1 g/kg body weight/day, about 10 mg/kg body weight/day to about 10 g/kg body weight/day, about 1 mg/kg body weight/day to about 10 mg/kg body weight/day, about 0.1 mg/kg body weight to about 10 mg/kg body weight, about 0.1 mg/kg body weight/day to about 10 mg/kg body weight/day, about 1 mg/kg body weight to about 1 g/kg body weight, about 0.01 mg/kg body weight to about 100 mg/kg body weight.

In other embodiments, the compounds of the present invention or other drug or prodrug product is administered in the range of about 10 ng to about 1 g per individual administration, about 10 ng to about 10 g per individual administration, about 10 ng to about 100 mg per individual administration, about 10 ng to about 1 mg per individual administration, about 10 ng to about 10 mg per individual administration, about 10 ng to about 100 mg per individual administration, about 10 ng to about 1000 mg per individual administration, about 10 ng to about 10,000 mg per individual administration, about 100 ng to about 1 mg per individual administration, about 100 ng to about 10 mg per individual administration, about 100 ng to about 100 mg per individual administration, about 100 ng to about 1 mg per individual administration, about 100 ng to about 10 mg per individual administration, about 100 ng to about 100 mg per individual administration, about 100 ng to about 1000 mg per individual administration, about 100 ng to about 10,000 mg per individual administration, about 1 mg to about 10 mg per individual administration, about 1 mg to about 100 mg per individual administration, about 1 mg to about 1.5 mg per individual administration, about 1 mg to about 5 mg per individual administration, about 1 mg to about 7.5 mg per individual administration, about 1 mg to about 10 mg per individual administration, about 1 mg to about 100 mg per individual administration, about 1 mg to about 1000 mg per individual administration about 1 mg to about 10,000 mg per individual administration, about 10 mg to about 100 mg per individual administration, about 10 mg to about 1 mg per individual administration, about 10 mg to about 10 mg per individual administration, about 10 mg to about 100 mg per individual administration, about 10 mg to about 1000 mg per individual administration, about 10 mg to about 10,000 mg per individual administration, about 100 mg to about 1 mg per individual administration, about 100 mg to about 10 mg per individual administration, about 100 mg to about 1 mg per individual administration, about 10 mg to about 1000 mg per individual administration, about 100 mg to about 10,000 mg per individual administration, about 1 mg to about 10 mg per individual administration, about 1 mg to about 100 mg per individual administration, about 1 mg to about 1000 mg per individual administration, about 1 mg to about 10,000 mg per individual administration, about 10 mg to about 100 mg per individual administration, about 10 mg to about 1000 mg per individual administration, about 10 mg to about 10,000 mg per individual administration, about 100 mg to about 1000 mg per individual administration, about 100 mg to about 10,000 mg per individual administration and about 1000 mg to about 10,000 mg per individual administration. Alternatively, the individual administration can be administered daily, BID, TID, QID, every 2, 3, 4, 5, 6 or 7 days, or every 1, 2, 3 or 4 weeks.

In other embodiments, the compounds of the present invention or other drug or prodrug product is administered in the range of about 10 ng to about 1 g per injection, about 10 ng to about 1 g per injection, about 100 ng to about 1 g per injection, about 1 ng to about 10 g per injection, about 10 ng to about 10 g per injection, about 100 ng to about 10 g per injection, about 1 ng to about 100 g per injection, about 10 ng to about 100 g per injection, about 100 ng to about 100 g per injection, about 1 ng to about 1 mg per injection, about 10 ng to about 1 mg per injection, about 100 ng to about 1 mg per injection, about 1 μg to about 1 mg per injection, about 10 μg to about 1 mg per injection, about 100 μg to about 1 mg per injection, about 1 μg to about 10 mg per injection, about 10 μg to about 100 mg per injection, about 10 μg to about 1 g per injection, about 100 μg to about 1 g per injection, about 100 μg to about 1 mg per injection, about 100 μg to about 10 mg per injection, about 1 mg to about 10 mg per injection, about 1 mg to about 5 mg per injection, about 1 mg to about 15 mg per injection, about 1 mg to about 20 mg per injection, about 1 mg to about 25 mg per injection, about 1 mg to about 30 mg per injection, about 1 mg to about 50 mg per injection, about 1 mg to about 100 mg per injection, about 1 mg to about 200 mg per injection, about 1 mg to about 500 mg per injection, about 1 mg to about 1 g per injection. Alternatively, injections of the proposed compounds and other drug products of the present invention can be injected daily, BID, TID, QID, every 2, 3, 4, 5, 6, or 7 days, or every 1, 2, 3 or 4 weeks.

In other embodiments, the amount of compound or other drug or prodrug product of the present invention can be administered at a dose of about 0.0001 mg, 0.0005 mg, 0.0006 mg, 0.001 mg, 0.003 mg, 0.006 mg, 0.01 mg, 0.03 mg, 0.06 mg, 0.1 mg, 0.3 mg, 0.6 mg, 1 mg, 2 mg, 3 mg, 5 mg, 6 mg, 10 mg, 30 mg, 60 mg, 70 mg, 80 mg, 90 mg, 100 mg, 300 mg, 600 mg, 700 mg, 800 mg, 900 mg, 1000 mg, 2000 mg, 3000 mg, 5000 mg, or 10,000 mg. As expected, the dosage will be dependent on the condition, size and age of the patient or subject.

In other aspects, the compounds proposed by the present invention and disclosed herein reduce the proliferation of a targeted disease (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of targeted disease, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds proposed by the present invention and disclosed herein reduce the signs and symptoms of inflammation by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the signs and symptoms of inflammation, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds proposed by the present invention and disclosed herein inhibit the activity of Proteinase 3 (PR3) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein inhibit the activity of Proteinase 3 (PR3), e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds proposed by the present invention and disclosed herein inhibit the activity of human neutrophil elastase (HNE) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein inhibit the activity of Human Neutrophil Elastase (HNE), e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds proposed by the present invention inhibit the activity of Proteinase 3 (PR3) and human neutrophil elastase (HNE) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein inhibit the activity of Proteinase 3 (PR3) and Human Neutrophil Elastase (HNE) by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds proposed by the present invention inhibit the binding activity of anti-neutrophil cytoplasmic antibodies (ANCA) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein inhibit the binding activity of anti-neutrophil cytoplasmic antibodies (ANCA) by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds proposed by the present invention inhibit the activity of Cathepsin G by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein inhibit the activity of Cathepsin G by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds proposed by the present invention suppress an undesired neutrophil immune response related to activation of the PR3 receptor by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein suppress an undesired neutrophil immune response related to activation of the PR3 receptor by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention suppress an undesired neutrophil immune response related to activation of the HNE receptor by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein suppress an undesired neutrophil immune response related to activation of the HNE receptor by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds proposed of the present invention suppress an undesired neutrophil immune response related to activation of the Cathepsin G receptor by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein suppress an undesired neutrophil immune response related to activation of the Cathepsin G receptor by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention suppress an undesired neutrophil immune response related to activation of the PR3 and Cathepsin G receptor by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein suppress an undesired neutrophil immune response related to activation of the PR3 and Cathepsin G receptor by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds suppress an undesired neutrophil immune response related to activation of the PR3 and HNE receptor by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein suppress an undesired neutrophil immune response related to activation of the PR3 and HNE receptor by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention suppress an undesired neutrophil immune response related to activation of the HNE and Cathepsin G receptor by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein suppress an undesired neutrophil immune response related to activation of the HNE and Cathepsin G receptor by, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds reduce the proliferation of Neutrophil-Driven Inflammatory Non-CF Bronchiectasis (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of Neutrophil-Driven Inflammatory Non-CF Bronchiectasis, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds reduce the proliferation of Neutrophil-Driven Inflammatory Cystic Fibrosis (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of Neutrophil-Driven Inflammatory Cystic Fibrosis, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention reduce the proliferation of Primary Sclerosing Cholangitis (PSC) (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of Primary Sclerosing Cholangitis (PSC), e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention reduce the proliferation of Granulomatosis with Polyangiitis (GPA) (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of Granulomatosis with Polyangiitis (GPA), e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention reduce the proliferation of PR3-ANCA Positive Ulcerative Colitis (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of PR3-ANCA Positive Ulcerative Colitis, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention reduce the proliferation of Alpha-1 Antitrypsin Deficiency (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of Alpha-1 Antitrypsin Deficiency, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention reduce the signs and symptoms of cystic fibrosis by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the signs and symptoms of cystic fibrosis, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention reduce the proliferation of inflammatory bowel disease (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of inflammatory bowel disease, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention reduce the proliferation of Ulcerative Colitis (e.g., signs and symptoms) by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the proliferation of Ulcerative Colitis, e.g., about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects, the compounds of the present invention reduce the signs and symptoms of Chronic Obstructive Pulmonary Disease (COPD), e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, the compounds proposed by the present invention and disclosed herein reduce the signs and symptoms of Chronic Obstructive Pulmonary Disease (COPD), e.g., by about 1% to about 5%, about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

The present invention further provides inhibition of the binding anti-neutrophil cytoplasmic antibodies (ANCA) to PR3. Such cytoplasmic antibodies contemplated by the invention include: cANCA antibodies, cytoplasmic neutrophil antibodies, anticytoplasmic autoantibodies, cytoplasmic antinuclear antibodies (ANA), and all other art-recognized equivalents.

The present invention further contemplates enhanced binding efficiency of small functional groups (OH, F, NH2) on appendages and terminal aromatic groups within the S1/S′ pocket. Terminal aromatic groups contemplated by the present invention include: hydrocarbons, benzenoids, non-benzenoids, polynuclear aromatic hydrocarbons, monocyclic aromatic hydrocarbons, polycyclic aromatic hydrocarbons, and all other art-recognized equivalents. Small functional groups contemplated by the invention include, but are not limited to: stereoisomers, geometric isomers, carbonyls, hydroxyls, phenols, ethers, alkanes, alkyl halides, alcohols, primary alcohols, secondary alcohols, tertiary alcohols, aldehydes, ketones, carboxylic acids, esters, amides, primary amines, secondary amines, tertiary amines, nitriles, carboxylate ions, carboxylate salts, amino acids, alkenes, trans-alkenes, cis-alkenes, and all components of the aforementioned groups and all art-recognized equivalents.

A salt bridge is contemplated within the present disclosure to allow bonding to Asp61, however other contemplations of non-covalent interactions for ionic bonding are suitable for the purposes of the present invention. All alternative and art-recognized equivalents to salt bridges are also suitable for the purposes of the present invention. A salt bridge or any functional art-recognized equivalent may also be used to allow bonding to Lys99 and Trp218, as well as any art-recognized and conceivable combination of the above.

A pharmaceutical composition and/or compound disclosed herein can include a solvent, emulsion or other diluent in an amount sufficient to dissolve a pharmaceutical composition disclosed herein. In other aspects of this embodiment, a pharmaceutical composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v).

In other aspects of this embodiment, a pharmaceutical composition and/or compound disclosed herein may include a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a pharmaceutical composition and/or compound disclosed herein can be of any concentration desired. In an aspect of this embodiment, the final concentration of a pharmaceutical composition and/or compound in a pharmaceutical composition may be a therapeutically effective amount. In other aspects of this embodiment, the final concentration of a pharmaceutical composition in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL or at least 500 mg/mL. In other aspects of this embodiment, the final concentration of a pharmaceutical composition in a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Dosing can be single dosage or cumulative (serial dosing), and can be readily determined by one skilled in the art. For instance, treatment of an ailment (e.g., GPA) can comprise a one-time administration of an effective dose of a pharmaceutical composition disclosed herein. Alternatively, treatment may include multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms and/or the chemotherapy treatment plan. For example, an effective dose of a pharmaceutical composition disclosed herein can be administered to an individual once immediately after a single chemotherapy treatment. Alternatively, it can be administered daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

In an embodiment, a formulation and/or compound as described herein can be administered by any suitable route, specifically by parenteral (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary, without limitation, with the composition used for therapy, the purpose of the therapy, and the subject being treated. Single or multiple administrations can be carried out, without limitation, the dose level and pattern being selected by the treating physician. Suitable dosage formulations and methods of administering the agents are known in the art.

In various embodiments, a formulation and/or compound can include one or more preservatives and/or additives known in the art. Similarly, a formulation can further be formulated into any of various known delivery formulations. For example, in an embodiment, a formulation can include, surfactants, adjuvant, biodegradable polymers, hydrogels, etc., such optional components, their chemical and functional characteristics are known in the art. Similarly known in the art are formulations that facilitate rapid, sustained or delayed release of the bioactive agents after administration. A formulation as described can be produced to include these or other formulation components known in the art.

The formulations and compounds as described herein can be used in the manufacture of medicaments and for the treatment of humans and other animals by administration in accordance with conventional procedures.

In an embodiment, the pH of the pharmaceutical formulation and/or compound is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9.

In an embodiment, the pH of the pharmaceutical formulation and/or compound is from about 3 to about 9, about 4 to about 19, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

### EXAMPLES

### Example 1: Modification of Human PR3 Structure

All ligands were prepared with LIGPREP from SMILES using OPLS3 force field modified using EPIK. Molecules were desalted and featured all possible tautomers between pH 5-9. Any chiral centers were held fixed. The reported human PR3 crystal structure (PDB 1D 1FUJ) was used to prepare a receptor grid for docking study.

Human PR3 protein structure first was modified using Maestro Protein Preparation Wizard selecting default values. Waters at 5 Å from heteroatoms were eliminated. The protonation states of side chains were modified with EPIK between pH 5-9. Positions of hydrogen bonds and torsion angles were refined prior to initiation, including the addition of missing side chains. Disulfide bonds were considered. Water orientations were sampled at pH 7. A search grid of 8000 Å³ (20×20×20 Å) was centered on location of binding pocket (the S1 pocket near Ile190 and Phe192, Catalytic Ser195, Ser214 and S′ pocket: Ala152, val154), for docking. Glide docking was performed.

Compounds were evaluated on docking score, E_{model} score, and Glide Score. Scores were compared versus known control compounds. MM-GBSA method in Prime was used to rank order the docked pose of compounds. These poses were taken as inputs for the energy minimization of the protein-ligand complexes (E_{complex}), the free protein (Eₚᵣₒₜₑᵢₙ), and the free ligands (E_{ligand}). The binding free energy ΔG_{bind} was determined according to the following equation: ΔG_{bind}=E_{complex} (minimized)−E_{ligand} (minimized)−E_{receptor} (minimized).

Compound characteristics and predicted physiochemical ADME/Tox properties were calculated and reported using Qikprop (Schrödinger software) for the enumerated compounds. Predicted blood-brain barrier (BBB) and cell membrane permeability, lipophilicity, aqueous solubility, drug-likeness (Lipinski's rules) and number of metabolic sites were reported.

### Example 2

### Selectivity Assessment Against Human Neutrophil Elastase

Selectivity assessment against human neutrophil elastase (HNE) was completed for each round of enumerated compounds using HNE crystal structure (PDB ID 1PPF). Receptor preparation was followed the same as PR3. Followed by GBSA calculations to rank order compounds.

Criteria for selection of hit compounds included: docking scores, MM-GBSA scores, and intermolecular interactions within the amino acid residues of the binding pocket of the PR3.

Ligand designer (Schrödinger Suite) and medicinal chemist's designs were utilized: “Ligand receptor interactions,” “bioisoster replacement,” “attach R-group” and “isostere screening” (throughout the compound) were attempted for different atoms on the scaffolds which resulted in 192 suggested compounds (8 compounds were Canem suggestions).

Docking was performed for the suggested enumerated compounds against human PR3, followed by MM-GBSA calculations (on 30% top-scored compounds) to rank order them.

### Example 3

### Predicting Ligand Binding Free Energies

A compiled list of 192 enumerated compounds based on their predicted ligand binding free energies and predicted ADME/tox (log P, cell permeability, aqueous solubility, etc.) were examined. Based on the selectivity assessment docking results, preserving compound salt bridge and hydrogen bond and interactions to Asp61, Lys99, and Trp218 respectively are suggested as improving selectivity towards PR3—owing to the fact that residue 99 and 218 are Leu and Gly in HNE.

A second and a third set of enumerated compounds were designed to have more selectivity towards PR3 based on the previous findings and using the same receptor grid as the previous cycle (FIGS. 2A and 2B).

Based on the docking results, a third round of suggestions were scored similarly against both PR3 and HNE specially for the top-scored compounds. Lys99 hydrogen bond interactions were excluded. ADME/Tox properties were predicted for those compounds. All calculated values from Schrödinger suite (molecular docking, predicted biological and physical properties) were used to evaluate and determine the quality of ˜500 proposed structures. In selecting DCT-5 and MP-77, the following were identified as important features for activity against PR3 and HNE:
1) Salt bridge to Asp61 appears to be valuable.
2) Appendages (generally containing basic functions) can reach into S1 pocket resulting in additional peptide interactions.
3) Small functional groups (OH, F, NH2) on appendages and terminal aromatic groups can be valuable.
4) The central amide group (H bond with Hys57) can be exchanged with imidazole.
5) The imidazolidinone (left ring) can be exchanged with 5- and 6-membered aromatic rings.
6) The stereochemistry around chiral centers can be important.
7) Small changes in the structure can have significant impact on the modeling scores and properties of interest. But also shows an ability to develop a structure-activity relationship.

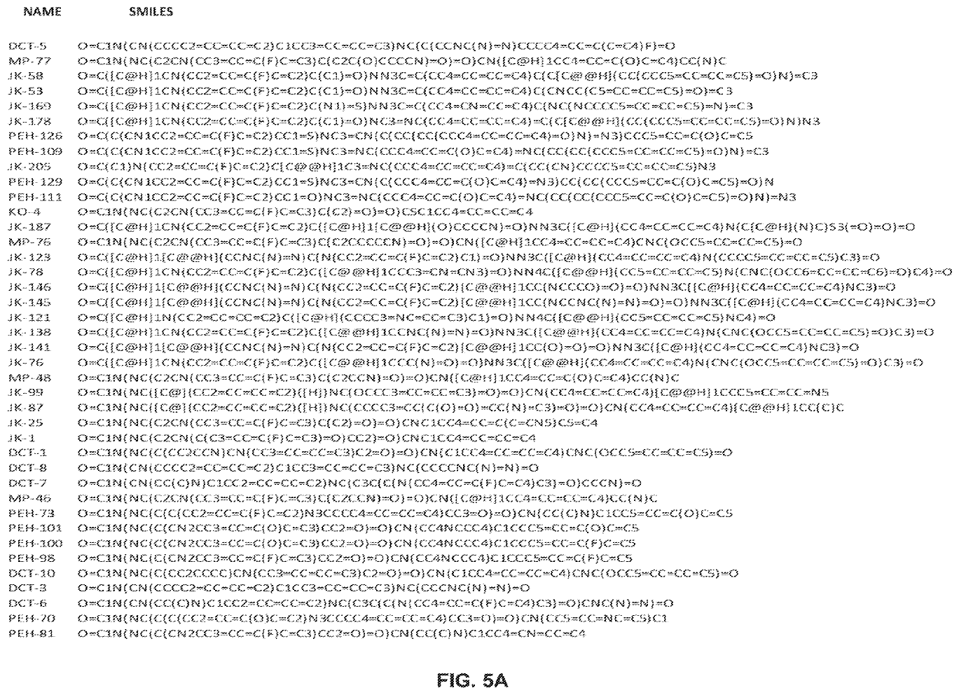

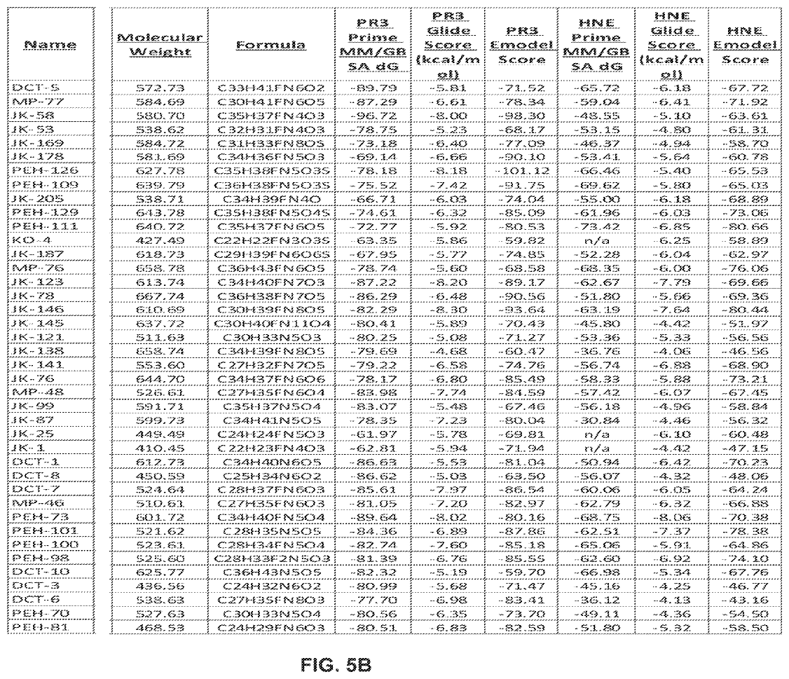

Structures like MP-77 explore the protein interactions of residue Asp61 and residues in the S1 pocket and provide very high modeling scores. Structures like DCT-5 provide an additional series coming from the common core imidazolidinone. Interactions with Asp61 and S1 pocket can be explored. DCT-5 resulted in high modeling scores. FIG. 5A is a table of compounds of the present invention with respective “SMILES.” SMILES is the “Simplified Molecular Input Line Entry System,” which is used to translate a chemical's three-dimensional structure into a string of symbols that is easily understood by computer software.

FIG. 5B lists results of in silico molecular docking studies and MM/GBSA analysis for each molecule. The complexes showed favorable docking score, glide energy, glide E_{model}, hydrogen bond and hydrophobic interactions between the active site residues of PR3/HNE and the compounds.

Molecular mechanics with generalized Born and surface area solvation (MM/GBSA) is a popular method to calculate the free energy of the binding of ligands to proteins. The 4th column lists the Prime MM/GBSA dG values for each molecule. GlideScore is an empirical scoring function that approximates the ligand binding free energy. The 5^{th} column lists the PR3 Glide Score (kcal/mol) for each molecule. The 6^{th} column lists the E_{model} Score for each molecule. Similarly, the values for HNE binding are listed for each molecule.

DCT5 and MP77 have a common core imidazolidinone with protein interactions on residue Asp61 and additional residues in the S1 pocket. DCT5 has binding per Prime MM/GBSA dG of approximately 89% for PR3 and 66% for HNE. MP77 has binding per Prime MM/GBSA dG of approximately 87% for PR3 and 59% for HNE. Projected CathG binding (not measured for either molecule) is expected to be very similar to HNE.

### Example 4

### Treatment of Alpha-1 Antitrypsin Deficiency

Alpha-1 antitrypsin deficiency (AATD) is an autosomal recessive disorder characterized by an increased risk for chronic obstructive pulmonary disease, (including emphysema, airflow obstruction and chronic bronchitis), liver disease and increased aminotransferase levels in the early period of life.

In this example, a patient is diagnosed with AATD after presenting symptoms related to difficulty breathing and mild jaundice. The diagnosis of AATD is confirmed by demonstration of a low concentration of alpha-1 antitrypsin (AAT) in serum, followed by detection of a functionally deficient AAT protein or detection of bi-allelic pathogenic variants in SERPINA1, the gene encoding alpha-1 antitrypsin. The patient receives conventional therapy (e.g., bronchodilators, inhaled corticosteroids, pulmonary rehabilitation and supplemental oxygen). Because the symptoms persist, the treating physician suggests periodic intravenous infusion of pooled human serum alpha-antitrypsin (i.e., augmentation therapy). This treatment leads to some improvement, but the patient returns to the clinic seeking an alternative treatment.

The treating physician administers a compound of Formula DCT-5A to the patient:

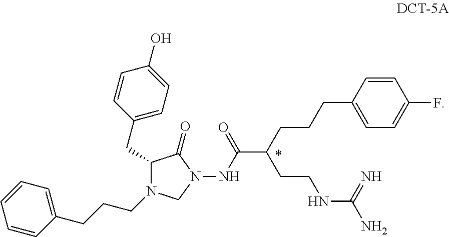 [SMILES: N=C(N)NCCC(CCCc1ccc(F)cc1)C(=O)NN1CN(CCCc2ccccc2)[C@H](Cc2ccc(O)cc2)C1=O]

The patient is administered the drug orally (i.e., 50 mg, once per day). Within two weeks, the signs/symptoms of AATD are resolved by about 80% and the patient appears in good health. The patient resumes the treatment and is periodically monitored for changes in her condition.

### Example 5

### Treatment of Severe Inflammation

Critically ill post-surgical, post-trauma and/or septic patients are characterized by severe inflammation. It has been proposed that dysfunction of neutrophils potentially increases the susceptibility to infections or can result in the inability to clear existing infections. In severe inflammation, neutrophils are rapidly activated, which affects their functional capacities, such as chemotaxis, phagocytosis, intra-cellular killing, NETosis, and their capacity to modulate adaptive immunity.

In this example, a patient is recovering from surgery following a traumatic vehicle accident. The treating physician anticipates further complications such as acute respiratory distress syndrome (ARDS) and (multiple) organ failure. To prevent this, the treating physician administers a compound of Formula DCT-5A to the patient. The patient is administered the drug intravenously (i.e., 5 mcg/minute for 1 hour). The patient is administered a second dose after 24 hours. Within two days, the patient shows signs of improvement. The patient resumes the treatment and is periodically monitored for changes in her condition.

### Example 6

Treatment of Granulomatosis with Polyangiitis (GPA)

Granulomatosis with polyangiitis (GPA) causes inflammation of the blood vessels in your nose, sinuses, throat, lungs and kidneys. Conventional treatments include drugs that suppress the immune system. Patients are monitoring for recurring symptoms

In this example, a patient complains of several symptoms including sinus pain, cough, fever, joint aches, blood in urine, and hearing loss. A physician diagnosis her with GPA after analyzing diagnostic markers.

The treating physician anticipates further complications such as acute respiratory distress syndrome (ARDS) and (multiple) organ failure. To prevent this, the treating physician administers a compound of Formula DCT-5A to the patient. The patient is administered the drug orally (i.e., 50 mg, once per day). Within one week, the signs/symptoms of AATD are resolved by about 90% and the patient appears in good health. The patient resumes the treatment and is periodically monitored for changes in her condition.

### Example 7

### Treatment of PR3 Positive Ulcerative Colitis

Serum anti-proteinase 3 antineutrophil cytoplasmic antibody (PR3-ANCA) is a disease-specific antibody against granulomatosis with polyangiitis. PR3-ANCA is a useful serological marker for disease severity in ulcerative colitis (UC).

In this example, a patient (male in his mid-thirties) is admitted to a hospital with cough and fever (over 39.0° C.). About one month prior, he had presented with fever (over 39.0° C.), diarrhea and bloody bowel discharge. Upon admission, his hemoglobin was 8.9 g/dl and the serum C-reactive protein level was 6.8 mg/dl. Colonoscopy revealed flare and erosions in the entire large intestine and the patient was diagnosed as having ulcerative colitis. Further, the patient tested positive for PR3-ANCA serological marker.

The treating physician administers a compound of Formula DCT-5A to the patient. The patient is administered the drug intravenously (i.e., 5 mcg/minute for 1 hour). The patient is administered a second dose after 24 hours. Within two days, the patient shows signs of improvement. Thereafter, the patient is administered the drug orally (50 mg per day). The patient resumes the treatment and is periodically monitored for changes in his condition.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

1. A compound of Formula DCT5A or an analog thereof:

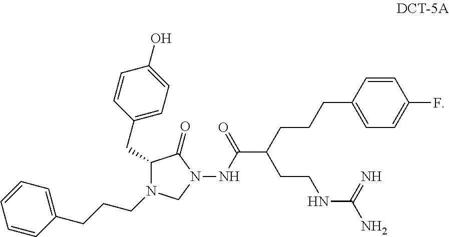 [SMILES: N=C(N)NCCC(CCCc1ccc(F)cc1)C(=O)NN1CN(CCCc2ccccc2)[C@H](Cc2ccc(O)cc2)C1=O]

2. The compound of claim 1, wherein the analog is selected from a compound of Formula DCT-5B and DCT5C:

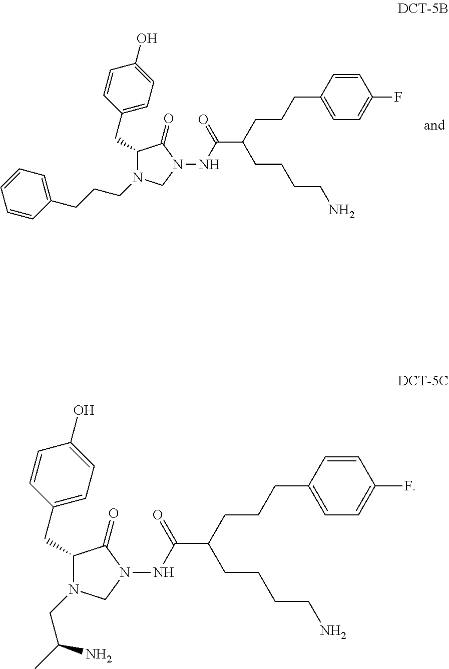 [SMILES: NCCCCC(CCCc1ccc(F)cc1)C(=O)NN1CN(CCCc2ccccc2)[C@H](Cc2ccc(O)cc2)C1=O ; C[C@H](N)CN1CN(NC(=O)C(CCCCN)CCCc2ccc(F)cc2)C(=O)[C@H]1Cc1ccc(O)cc1]

3. A method of treating an ailment in a subject, the method comprising administering a therapeutic amount of the compound of claim 1 to the subject.

4. The method of claim 3, wherein the ailment is an autoimmune or autoinflammatory disease.

5. The method of claim 3, wherein the ailment is one or more of ANCA-associated vasculitis, ulcerative colitis, systemic lupus, erythematosus, rheumatoid arthritis, gout and inflammatory bowel disease (IBD).

6.-7. (canceled)

8. The method of claim 3, wherein the ailment is alpha-1 antitrypsin deficiency.

9. The method of claim 3, further comprising administering an immunosuppressive agent.

10. The method of claim 9, wherein the immunosuppressive agent is one or more of cyclophosphamide, rituximab, methotrexate, azathioprine and a glucocorticoid.

11.-20. (canceled)

21. A method of inhibiting the activity of Proteinase 3 (PR3) and/or Human Neutrophil Elastase (HNE) in a subject in need thereof, the method comprising administering an effective amount of a compound selected from DCT-5A, DCT-5B, DCT-5C, MP-77, MP77B and MP-77C:

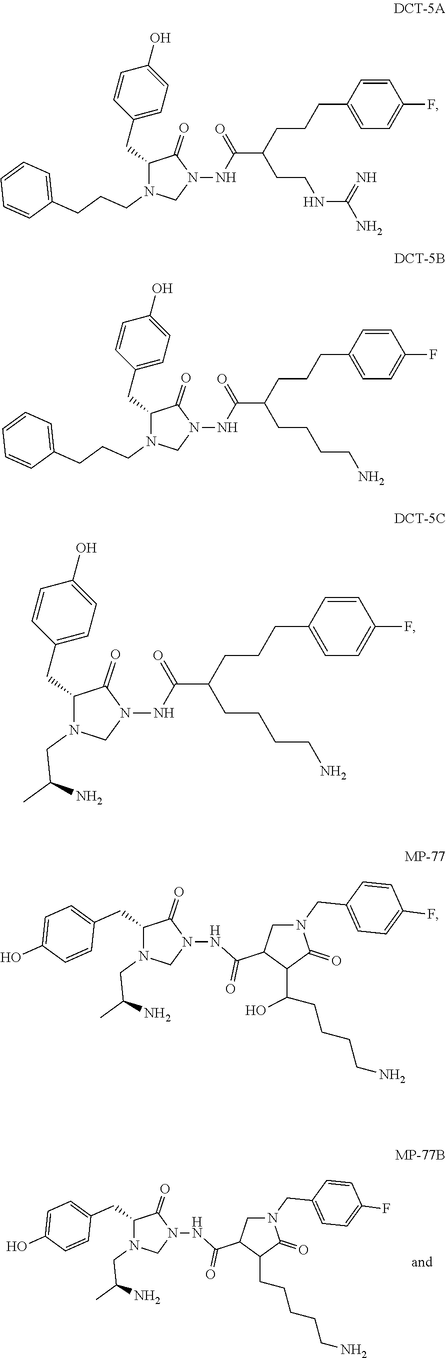 [SMILES: N=C(N)NCCC(CCCc1ccc(F)cc1)C(=O)NN1CN(CCCc2ccccc2)[C@H](Cc2ccc(O)cc2)C1=O ; NCCCCC(CCCc1ccc(F)cc1)C(=O)NN1CN(CCCc2ccccc2)[C@H](Cc2ccc(O)cc2)C1=O ; C[C@H](N)CN1CN(NC(=O)C(CCCCN)CCCc2ccc(F)cc2)C(=O)[C@H]1Cc1ccc(O)cc1 ; C[C@H](N)CN1CN(NC(=O)C2CN(Cc3ccc(F)cc3)C(=O)C2C(O)CCCCN)C(=O)[C@H]1Cc1ccc(O)cc1 ; C[C@H](N)CN1CN(NC(=O)C2CN(Cc3ccc(F)cc3)C(=O)C2CCCCCN)C(=O)[C@H]1Cc1ccc(O)cc1]

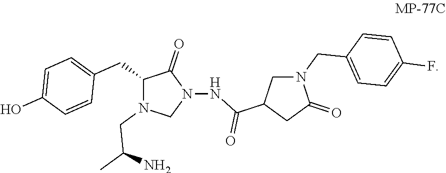 [SMILES: C[C@H](N)CN1CN(NC(=O)C2CC(=O)N(Cc3ccc(F)cc3)C2)C(=O)[C@H]1Cc1ccc(O)cc1]

22. The method according to claim 21, wherein the inhibiting the activity of PR3 comprises inhibition of binding anti-neutrophil cytoplasmic antibodies (ANCA).

23. The method according to claim 21, wherein the compound replaces the function of alpha-1 protease inhibitor and is utilized by inhibiting protease activity of human neutrophil elastase (HNE) and Proteinase 3 (PR3) in a patient.

24. The method according to claim 21, wherein the effective amount is from about 0.01 mg/kg to 10 mg/kg body weight of the patient or subject.

25. A compound of formula JK-58, JK-53, JK-169, JK-178, PEH-126, PEH-109, JK-205, PEH-129, PEH-111, KO-4, JK-187, JK-123, MP-76, JK-78, JK146, JK-145, JK121, JK138, JK141, JK-76, MP-46, JK-99, JK-87, JK-25, JK-1, PEH-70, DCT-1, DCT-8, DCT-7, MP-48, PEH-73, PEH-101, PEH-100, PEH-98, PEH-81, DCT-10, DCT-3 or DCT-6, a pharmaceutical salt, analog or pro-drug thereof:

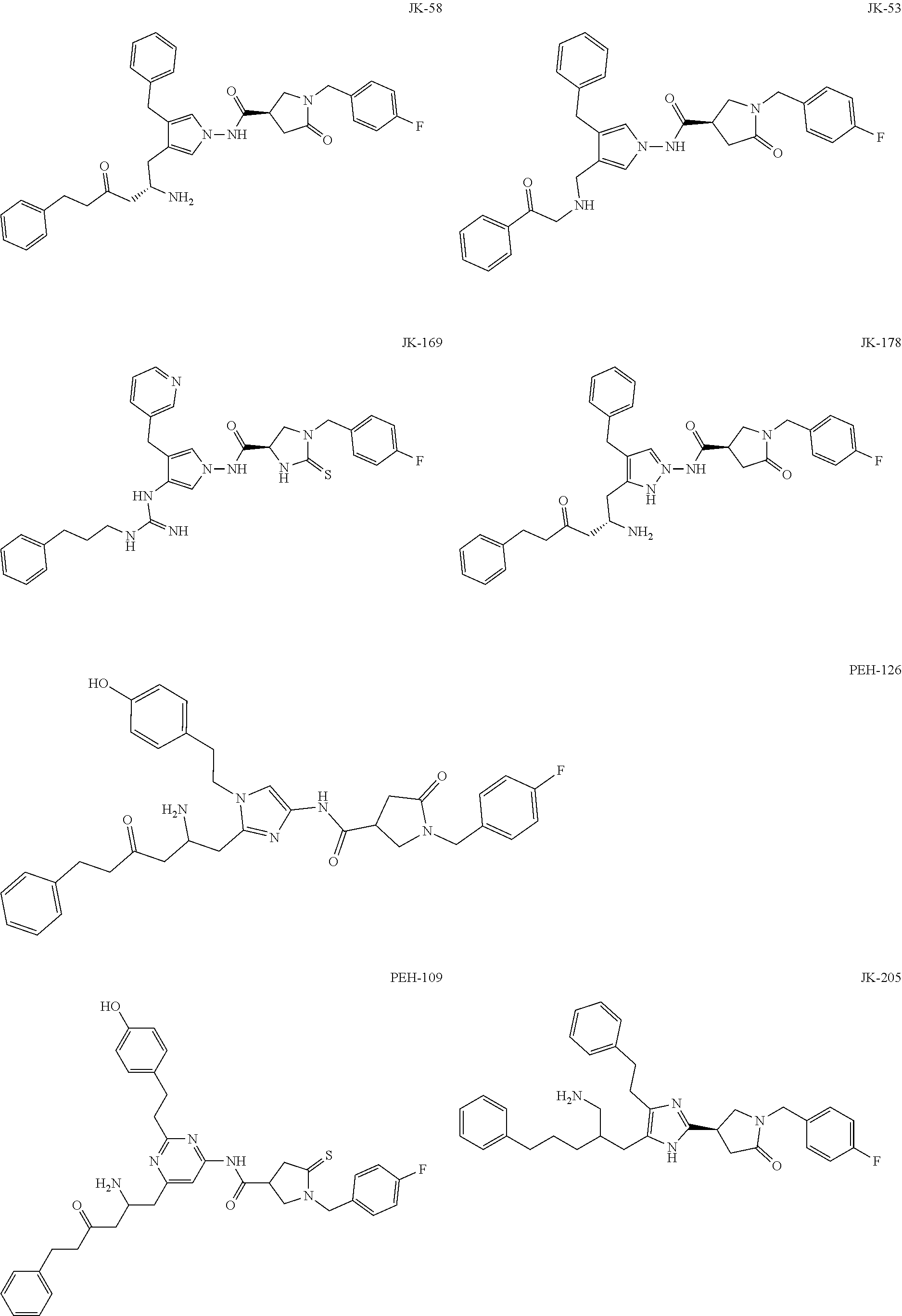

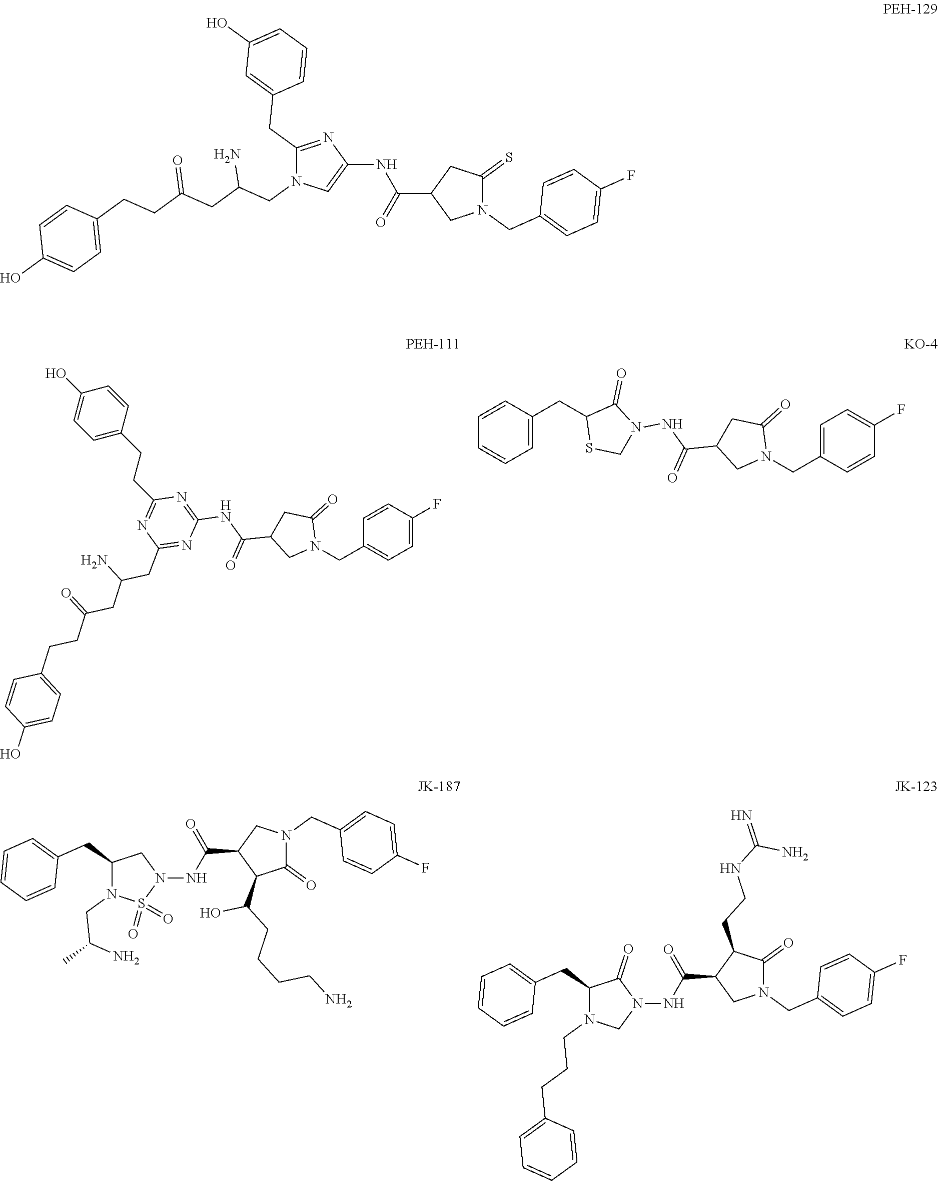 [SMILES: NC(CC(=O)CCc1ccc(O)cc1)Cn1cc(NC(=O)C2CC(=S)N(Cc3ccc(F)cc3)C2)nc1Cc1cccc(O)c1 ; NC(CC(=O)CCc1ccc(O)cc1)Cc1nc(CCc2ccc(O)cc2)nc(NC(=O)C2CC(=O)N(Cc3ccc(F)cc3)C2)n1 ; O=C(NN1CSC(Cc2ccccc2)C1=O)C1CC(=O)N(Cc2ccc(F)cc2)C1 ; C[C@@H](N)CN1[C@@H](Cc2ccccc2)CN(NC(=O)[C@H]2CN(Cc3ccc(F)cc3)C(=O)[C@H]2C(O)CCCCN)S1(=O)=O ; N=C(N)NCC[C@H]1C(=O)N(Cc2ccc(F)cc2)C[C@H]1C(=O)NN1CN(CCCc2ccccc2)[C@@H](Cc2ccccc2)C1=O]

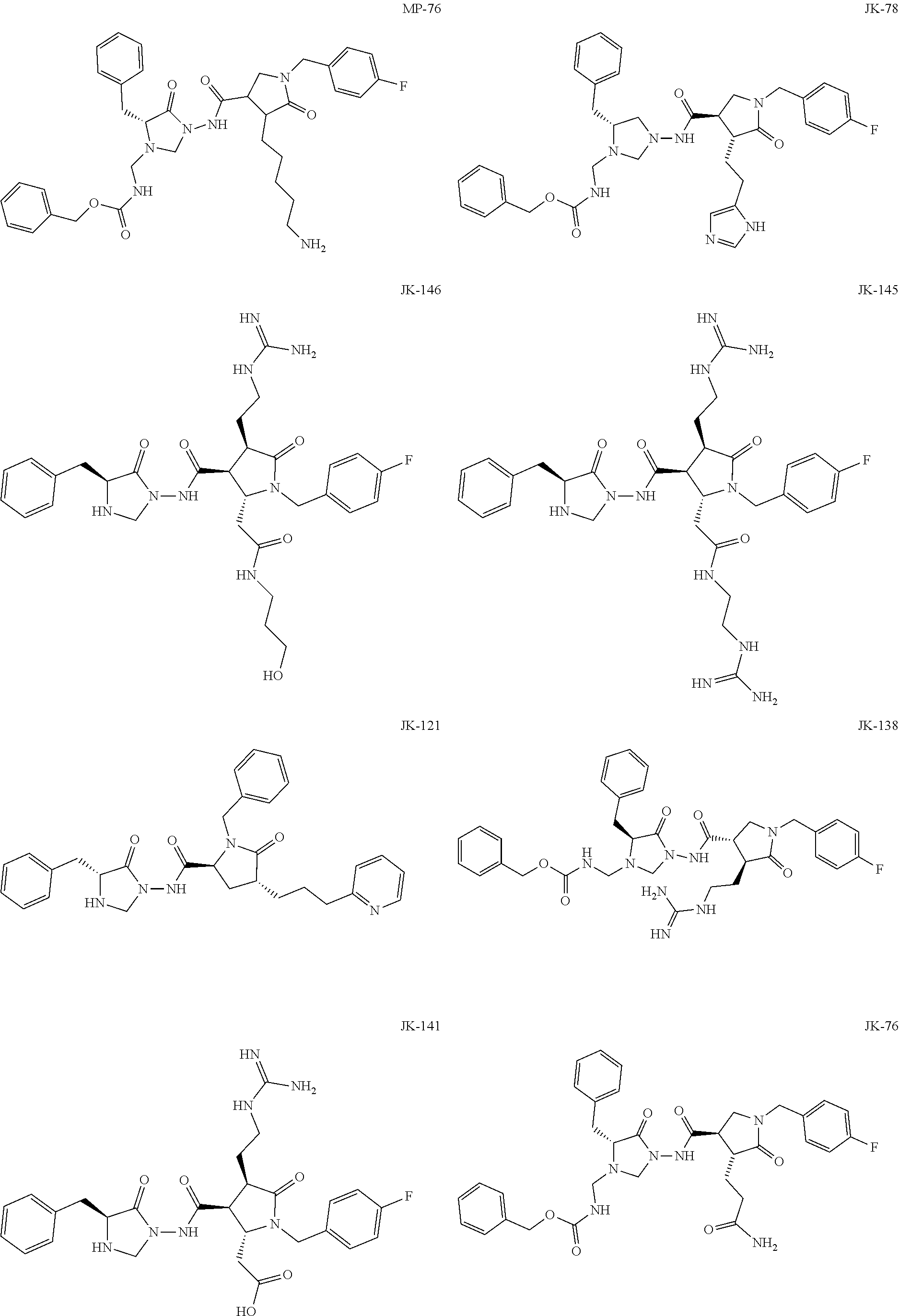

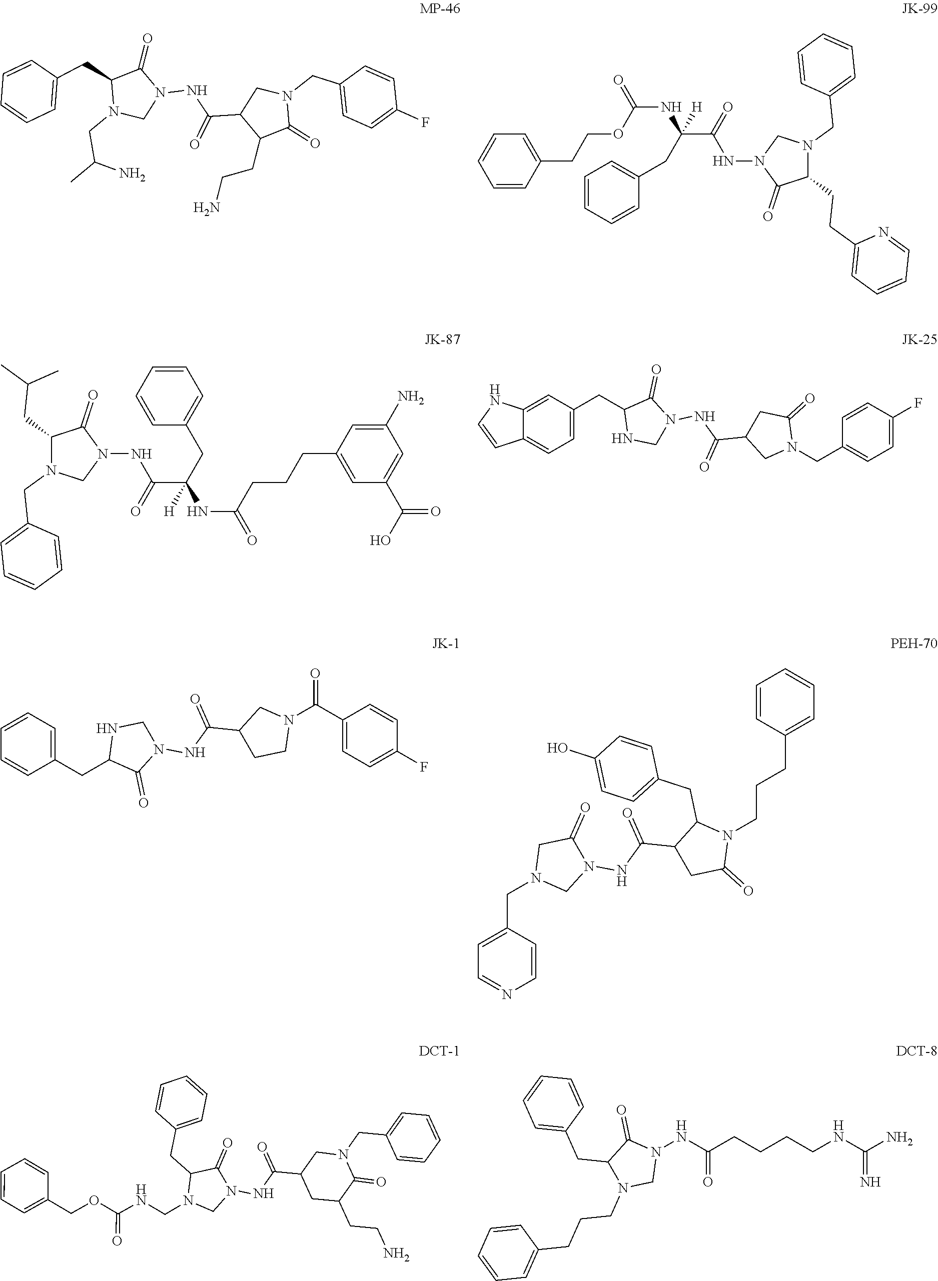

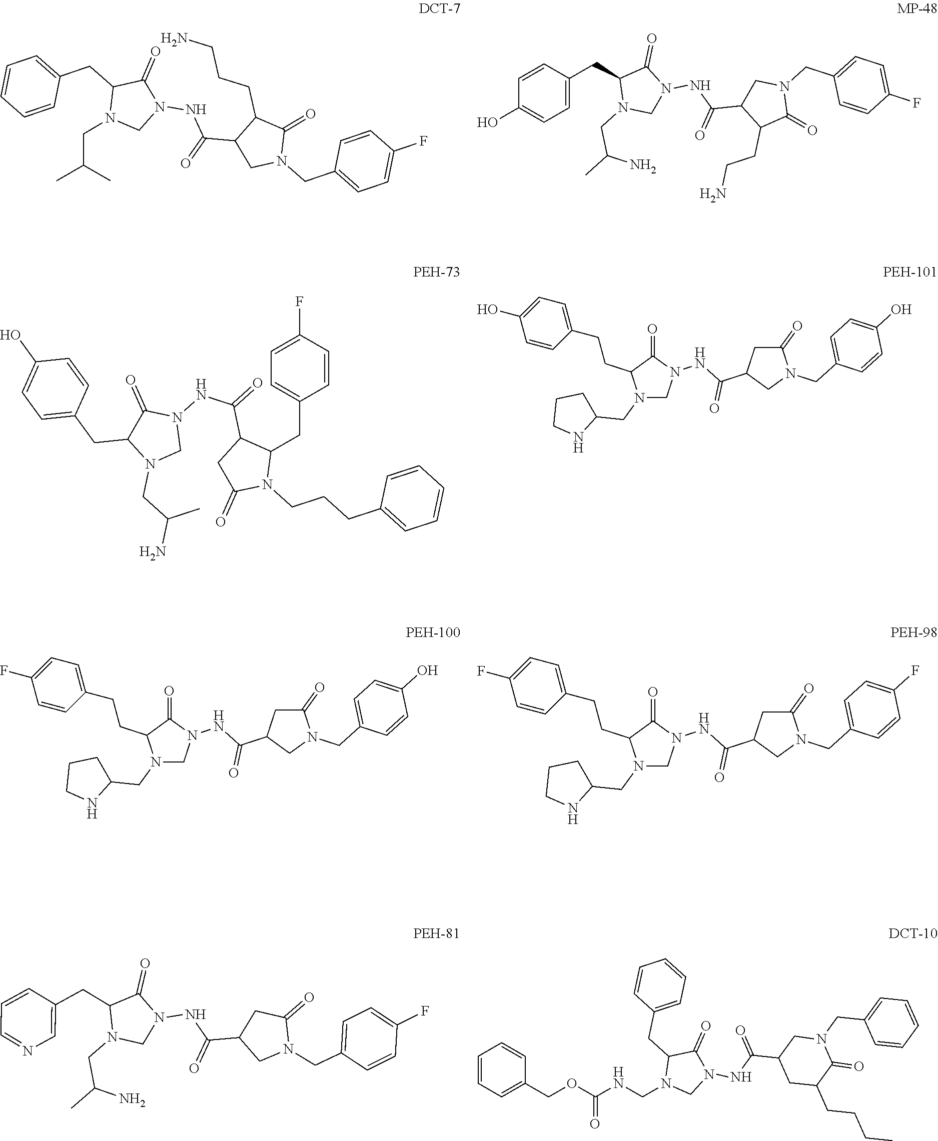

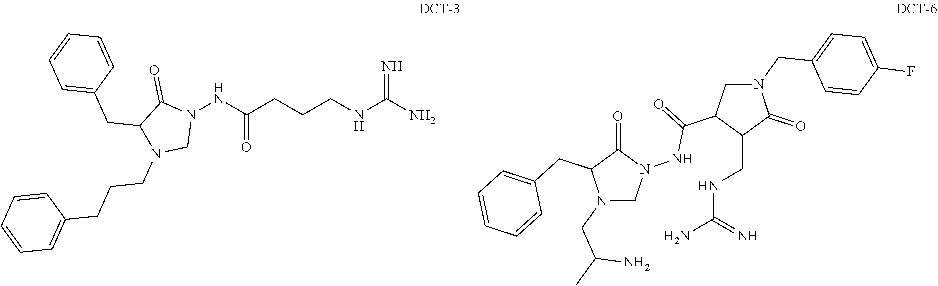 [SMILES: N=C(N)NCCCC(=O)NN1CN(CCCc2ccccc2)C(Cc2ccccc2)C1=O ; CC(N)CN1CN(NC(=O)C2CN(Cc3ccc(F)cc3)C(=O)C2CNC(=N)N)C(=O)C1Cc1ccccc1]

26. A method of inhibiting the activity of Proteinase 3 (PR3) and/or Human Neutrophil Elastase (HNE) in a patient in need thereof, comprising administering to the patient or subject an effective amount of a compound of claim 25.

27. A method of treating an ailment in a subject, the method comprising administering a therapeutic amount of a compound of claim 25 to the subject.

28. (canceled)

29. The method of claim 27, wherein the ailment is
an autoimmune or autoinflammatory disease selected from ANCA-associated vasculitis, ulcerative colitis, systemic lupus, erythematosus, rheumatoid arthritis, gout and inflammatory bowel disease (IBD).

30. The method of claim 27, wherein the ailment is one or more of cystic fibrosis, COPD, asthma or transfusion-related acute lung injury.

31. (canceled)

32. The method of claim 27, wherein the ailment is alpha-1 antitrypsin deficiency.

33. The method of claim 27, further comprising administering an immunosuppressive agent.

34. The method of claim 33, wherein the immunosuppressive agent is one or more of cyclophosphamide, rituximab, methotrexate, azathioprine and a glucocorticoid.

35. The method of claim 27, wherein the effective amount is from about 0.01 mg/kg to 10 mg/kg body weight of the patient or subject.
